(19) United States
(12) Patent Application Publication
(10) Pub. No.: US 2025/0176976 A1
(43) Pub. Date: Jun. 5, 2025

(54) PEDICLE ACCESS DEVICES, SYSTEMS AND METHODS

(71) Applicant: Relievant Medsystems, Inc., Edina, MN (US)

(72) Inventors: Brian W. Donovan, San Jose, CA (US); Neil Shigeo Sasaki, San Jose, CA (US); Benjamin David Parker, Oakland, CA (US); Michael Willink, Sunnyvale, CA (US); Phillip Michael Olsen, Plymouth, MN (US)

(21) Appl. No.: 18/963,978

(22) Filed: Nov. 29, 2024

Related U.S. Application Data
(60) Provisional application No. 63/604,775, filed on Nov. 30, 2023.

(51) Int. Cl. A61B 17/16; A61B 17/00; A61B 18/00; A61B 18/14

(52) U.S. Cl. CPC A61B 17/1671; A61B 18/148; A61B 2017/00367; A61B 2018/0044; A61B 2018/00577

(57) ABSTRACT

A medical device for forming a channel into a bone having an outer surface, includes an introducer tube having a distal end and a proximal end disposed in a longitudinal direction; a handle coupled with the proximal end of the introducer tube, wherein an internal channel is formed through the introducer tube and the handle, the internal channel configured to receive another longitudinal device from the handle to the distal end of the introducer tube; and an edge formed at the distal end of the introducer tube; a distance from the edge to a tip of the distal end of the introducer tube forming a distal end length, the distal end length being greater than 0, wherein the edge is configured to engage with the outer surface of the bone.

### CROSS REFERENCE TO RELATED APPLICATIONS

This application claims priority to U.S. Provisional Patent Application No. 63/604,775 entitled “PEDICLE ACCESS DEVICES, SYSTEMS AND METHODS,” filed Nov. 30, 2023, which is incorporated herewith in its entirety.

### FIELD

Described herein are various implementations of devices, systems and methods for facilitating introduction of instruments into a bone such as a vertebral body. The instruments may be used, for example, for accessing and/or for carrying out modulation (e.g., radiofrequency ablation) of one or more nerves within the vertebral body, one or more nerves innervating one or more endplates of the vertebral body, and/or one or more nerves innervating an intervertebral disc adjacent the vertebral body. In some instances, the instruments are adapted to be inserted through a pedicle (e.g., transpedicularly).

### BACKGROUND

Chronic back pain is a serious affliction that can greatly impact both physical and mental health. Back pain affects millions of people worldwide each year. Research has reported that low back pain and neck pain tops US health spending. Further, low back pain is a major cause for work-related disability. Back pain may arise from strained muscles, ligaments, or tendons in the back and/or structural problems with bones or spinal discs. Existing treatments for chronic back pain vary widely, including physical therapy and exercise, chiropractic treatments, injections, rest, and pharmacological therapy such as opioids, pain relievers or anti-inflammatory medications. Patients with severe back pain may get surgical intervention such as vertebral fusion, discectomy (e.g., total disc replacement), or disc repair. Existing treatments can be costly, addictive, temporary, ineffective, and/or can increase the pain or require long recovery times. In addition, existing treatments do not provide adequate relief for the majority of patients and only a small percentage are surgically eligible.

### SUMMARY

Applicant's existing technology (the INTRACEPT® procedure by Relievant Medsystems, Inc.) offers a safe and effective minimally invasive procedure that targets intraosseous nerves (e.g., the basivertebral nerve) for the relief of chronic low back pain. As disclosed herein, several embodiments provide additional modality and method of relief for patients and adjunct technologies.

In accordance with several embodiments, introduction of instruments is desired to be controlled so as to prevent, or reduce the likelihood of, inadvertent over-insertion, or insertion to a greater depth than desired within a bone (e.g., vertebral body). In some embodiments, introducer instruments are inserted within the bone (e.g. via malleting, pressing, pushing) and the introducer instruments can include various features, mechanisms or designs to facilitate the prevention or reduction in the likelihood of inadvertent or undesired over-insertion. For example, the introducer instruments can include surface features, depth stop features, etc. to impede or prevent further movement within the bone (e.g., vertebral body and/or pedicle). In other examples, separate mechanisms or devices may interact or interface with the introducer instruments to prevent or limit advancement of the introducer instruments.

In accordance with some embodiments, an introducer cannula for forming a channel into a bone having an outer surface comprises an introducer tube having a distal end and a proximal end disposed in a longitudinal direction, a handle coupled with the proximal end of the introducer tube. An internal channel is formed through the introducer tube and the handle. The internal channel is configured to receive a longitudinal device from the handle through the distal end of the introducer tube. An edge or other depth stop feature is formed at the distal end of the introducer tube, forming a distal end length from the edge to a tip of the distal end of the introducer tube. The edge is configured to engage with the outer surface of the bone.

In some embodiments, the edge is a ringed edge formed around the lateral surface of the introducer tube. In some embodiments, the edge is formed on an enlarged portion of the introducer tube. In some embodiments, the edge is formed on a block coupled to the introducer tube.

In some embodiments, the edge is formed by a distal end of a hypotube which is sleeved on the introducer tube, wherein a proximal end of the hypotube is coupled to the handle and the distal end length is adjustable by moving the hypotube longitudinally.

In some embodiments, the edge has a blunt curvature for smooth and gentle engagement with the bone. In some embodiments, the edge is partially bullet shaped. In some embodiments, the edge is beveled.

In some embodiments, longitudinal movement of the hypotube is achieved by rotating a wheel feature or other rotational mechanism coupled to the handle, the wheel feature rotating around the introducer tube.

In some embodiments, the handle comprises an internal channel formed in the longitudinal direction. The internal channel has an internal thread. The proximal end of the hypotube has an external thread formed thereon configured to mate with the internal thread formed in the handle, wherein the proximal end of the hypotube is coupled to the wheel feature in a manner so that turning the wheel feature causes the hypotube to rotate but allows the hypotube to move freely longitudinally. In some embodiments, the external thread is formed on the hypotube by insert molding.

In some embodiments, the handle comprises a protruded portion disposed in the longitudinal direction with an external thread formed thereon. The hypotube is coupled with a wheel feature, which has a hollowed extension disposed in the longitudinal direction with an internal thread formed therein. The external thread on the protruded portion of the handle is configured to mate with the internal thread formed in the hollow extension of the wheel feature.

In some embodiments, the coupling between the hypotube and the wheel feature is a permanent attachment. As such, rotating the wheel feature may cause the hypotube to move in the longitudinal direction. In some embodiments, the hypotube includes a feature that only allows movement in one direction when enabled. In some embodiments, the attachment between the hypotube and the wheel feature is accomplished by insert molding. In some embodiments, the attachment between the hypotube and the wheel feature is accomplished by interference fit features. In some embodiments, the attachment between the hypotube and the wheel feature is accomplished by use of one or more adhesives.

In some embodiments, the coupling between the hypotube and the wheel feature allows the hypotube to move in the longitudinal direction, wherein a resilient ring is disposed inside the hollowed space of the hollowed extension of the feature and outside of the hypotube. As such, rotating the wheel feature in one direction causes the resilient ring to be squeezed against the outer surface of the hypotube to stop the movement of the hypotube in the longitudinal direction.

In accordance, with several embodiments, an introducer tool (e.g. introducer cannula includes an adjustable length hypotube that allows an operator to access bone (e.g., the vertebral body). The introducer tool includes a distal end having a blunted bullet shape tip that allows for flush placement on uneven surfaces of the pedicle or other bone. The distal end may alternatively include a beveled bullet shape tip or plastically deformed waves.

In some embodiments, a proximal mechanism is adapted or configured to cause the adjustable hypotube to move distally and/or proximally. In some embodiments, a threaded insert may be overmolded on the hypotube that can be screwed or unscrewed to allow for contact of the distal end with the patient (e.g., pedicle or vertebral body). In some embodiments, a Tuohy-Borst mechanism is employed that applies friction to the hypotube to hold it in place once deployed. The Tuohy-Borst mechanism can be implemented in a handle of the introducer tool (e.g., introducer cannula) or can be free floating as an attachment similar to a collar on a rod or shaft. In some embodiments, the proximal mechanism is a vertical tooth mechanism that allows for a flexible member that can be accessed from the outside to slide distally with force that does not retract backwards because of gear teeth.

In some embodiments, a distal end of the hypotube of the introducer tool can include one or more features, such as teeth, knurling, media-blasted surfaces, etc. to provide friction to advancement of the introducer tool (e.g., introducer cannula).

In some embodiments, a medical system for performing radiofrequency (RF) ablation of one or more nerves within a vertebral body comprises an introducer cannula as described above, a straight stylet, the straight style is configured to be received into the internal channel formed in the introducer cannula, a curved cannula assembly comprising a curved cannula and a J-stylet, wherein the curved cannula comprises a second internal channel formed therethrough configured to receive another longitudinal device. The curved cannula assembly is configured to be received into the internal channel formed in the device. The medical system can further comprise a radiofrequency (RF) probe, the RF probe configured to be received into the second internal channel of the curved cannula. The RF probe may be configured to modulate one or more nerves within bone (e.g., vertebral body) to treat, for example, chronic low back pain.

In accordance with several embodiments, a kit for accessing and ablating one or more nerves within a vertebral body includes an introducer assembly including an introducer cannula and an introducer stylet, the introducer assembly adapted to form a straight access path through a pedicle to a border of a vertebral body. The introducer cannula includes a pedicle stop mechanism. The curved cannula assembly includes a curved cannula with a pre-curved distal end and a J-stylet with a pre-curved distal end corresponding to the pre-curved distal end of the curved cannula. The curved cannula assembly is adapted to form a curved path from the straight access path toward a predetermined target treatment region within a cancellous bone portion of a vertebral body. The kit also includes a radiofrequency ablation probe.

The pedicle stop mechanism may comprise, for example, an adjustable hypotube comprising a pedicle stop configured to engage with bone to prevent or limit further advancement of the hypotube. The pedicle stop mechanism may comprise any of the other pedicle stop mechanisms described herein (e.g., knurled surfaces, enlarged portions of the introducer tool, ratchet mechanism, gear wheels, Tuohy-Borst mechanisms, etc.).

The kit may further include a template (such as described herein) to facilitate guided access of the introducer assembly through a pedicle.

Devices and techniques are also contemplated to facilitate guided access into a pedicle. For example, mechanical, electronic, and/or software devices may be used to help physicians navigate pedicle access safely and more efficiently. Mechanical devices may be used to facilitate guided access as well as prevent against over-insertion through a pedicle or other bone access pathway. Mechanical devices may include, for example, adhesive stickers with radiopaque markings adapted to be aligned with anatomical landmarks (e.g., landmarks of a spine or vertebra), adhesive devices adapted to be adhered to a patient and having extensions or projections extending or projecting upward and outward from the adhesive portion at pre-set or adjustable angles and having a lumen adapted to allow introducer instruments to be inserted therethrough at a desired angle to facilitate guided access, or similar devices adapted to be coupled to an introducer cannula that include radiographic features that can be viewed on fluoroscopy to facilitate access along a particular trajectory (e.g., based on real-time imaging or pre-procedural imaging conducted in a pre-procedural planning session). Electronic and/or software devices may involve pre-planning that includes registration and establishment of coordinate systems and calculations of trajectories (e.g., azimuth angle, elevation angle, x, y, z coordinates) that can be used during a procedure to facilitate guided bone access (e.g., via laser projections or image-guided navigation).

In accordance with several embodiments, a device for facilitating guided pedicle access comprises an adhesive-backed template or sticker that has a surface pattern of radiopaque markings that allows an operator to align the markings with certain anatomical landmarks (e.g., endplate, lateral borders of the pedicle) along a spine of a patient, thereby providing a template for an incision point.

In some embodiments, the radiopaque markings comprise a layer of radiopaque ink or metal deposition to create the pattern to allow for viewing radiographically during the procedure.

The pattern may account for varying tissue thickness to reduce time in determining proper entry position.

In some embodiments, the device is a single-use sticker.

In some embodiments, the device is reusable.

In some embodiments, the device is custom-printed for a particular patient based on pre-operative imaging of at least a portion of the spine of the patient and/or planning software.

In some embodiments, a kit comprising a variety of any of the above devices is provided to allow for selection during procedural use.

In accordance with several embodiments, a method of facilitating guided pedicle access comprises using pre-operative planning software to calculate an optimal pedicle access trajectory for procedural use (e.g., for accessing and modulating one or more nerves within a vertebral body).

In some embodiments, the software uses image recognition or machine learning to find the optimal pedicle access trajectory.

In some embodiments, a trajectory is adjusted manually by an operator to find the optimal pedicle access trajectory.

In some embodiments, the software is programmed to output a coordinate system from certain spine landmarks (e.g., the L5 spinous process) to provide x, y, elevation angle and/or azimuth angle that an operator can then use to facilitate guided pedicle access for a patient.

In accordance with several embodiments, a method of facilitating guided pedicle access includes determining a vertebral body height based on pre-operative measurements. The determining of the vertebral body height may be performed manually or automatically via a software algorithm. The method also includes applying a template overlay adhesive to a back of a patient by aligning one or more radiopaque markings with one or more anatomical landmarks of a spine of the patient. The method further includes identifying a skin entry point positioned along a radiopaque trajectory line of the template overlay adhesive that properly dissects a pedicle projection. The method also includes inserting and advancing an introducer tool (e.g., introducer assembly including an introducer cannula and introducer stylet) through the skin entry point and through a pedicle while matching a trajectory angle of the introducer tool with the radiopaque trajectory line under fluoroscopic guidance.

For purposes of summarizing the disclosure, certain aspects, advantages, and novel features are discussed herein. It is to be understood that not necessarily all such aspects, advantages, or features will be embodied in any particular embodiment of the disclosure, and an artisan would recognize from the disclosure herein a myriad of combinations of such aspects, advantages, or features. The embodiments disclosed herein may be embodied or carried out in a manner that achieves or optimizes one advantage or group of advantages as taught or suggested herein without necessarily achieving other advantages as may be taught or suggested herein. The systems and methods of the disclosure each have several innovative aspects, no single one of which is solely responsible for the desirable attributes disclosed herein.

The devices and methods summarized above and set forth in further detail below describe certain actions taken by a practitioner; however, it should be understood that they can also include the instruction of those actions by another party. Thus, actions such as “ablating” or “advancing” include “instructing the ablating or advancing.” Further aspects of embodiments of the disclosure will be discussed in the following portions of the specification. With respect to the drawings, elements from one figure may be combined with elements from the other figures.

### BRIEF DESCRIPTION OF THE DRAWINGS

The following drawings are for illustrative purposes only and show non-limiting embodiments. Features from different figures may be combined in several embodiments. It should be understood that the figures are not necessarily drawn to scale. Distances, angles, etc. are merely illustrative and do not necessarily bear an exact relationship to actual dimensions and layout of the devices illustrated.

FIG. 1 illustrates an example set of access tools and a treatment device configured to access and modulate (e.g., ablate, stimulate) a nerve within a vertebral body.

FIGS. 2-5 are schematic views illustrating steps of an example treatment procedure for accessing a vertebral body and modulating (e.g., ablating) a nerve within the vertebral body.

FIG. 6 illustrates two example embodiments of introducer cannulas with features for slowing down or impeding the advancement thereof into a pedicle.

FIGS. 7-9 are schematic views illustrating example embodiments of introducer cannulas having distal ends with modified shapes to slowdown to stop further advancement thereof.

FIG. 10 shows an example embodiment of an introducer cannula comprising an adjustable hypotube sleeved over an introducer tube, the hypotube having a distal end forming an adjustable length between the distal end and a distal tip of the introducer tube.

FIGS. 11A-15C shows example embodiments of introducer cannulas comprising mechanisms at proximal handles for extending or retracting the hypotube of FIG. 10.

FIGS. 16-21 are schematic views illustrating more example embodiments of introducer cannulas with features for impeding or stopping the advancement thereof into the pedicle.

FIGS. 22A-24B illustrate example embodiments of devices and techniques to facilitate pedicle access to a vertebral body.

### DETAILED DESCRIPTION

Damaged or degenerated vertebral endplates may be significant sources of chronic low back pain. The pain may be transmitted from the superior and interior endplates through one or more nerves (e.g., a basivertebral nerve, which enters through an opening on the posterior wall of the vertebral body and branches near the center of the vertebral body, sending nerves to innervate the superior and inferior endplates). Therefore, this type of back pain may be deemed to originate from the vertebrae. The Applicant's research and clinical trials have established that such back pain originating from the vertebrae can be treated by RF ablation.

In some implementations, discogenic back pain may also (additionally or alternatively) be treated by applying ablative RF energy within the vertebral body.

### A. Example Access Tools and Treatment Device

Procedures have been developed by the Applicant to target one or more nerves within the vertebral body (e.g., the basivertebral nerve) so as to relieve, for example, chronic low back pain, and to improve functioning. In some embodiments, a radiofrequency (RF) ablation probe is introduced into a selected vertebral body to perform such a minimal invasive RF ablation or other modulation procedure. To modulate the whole nerve within the vertebral body, a small area near the center of the vertebral body where the basivertebral nerve branches off is usually pre-identified and targeted. The targeted area may be in a posterior half of the vertebral body (e.g., about 10-50% of the posterior to anterior wall distance at the posterior side).

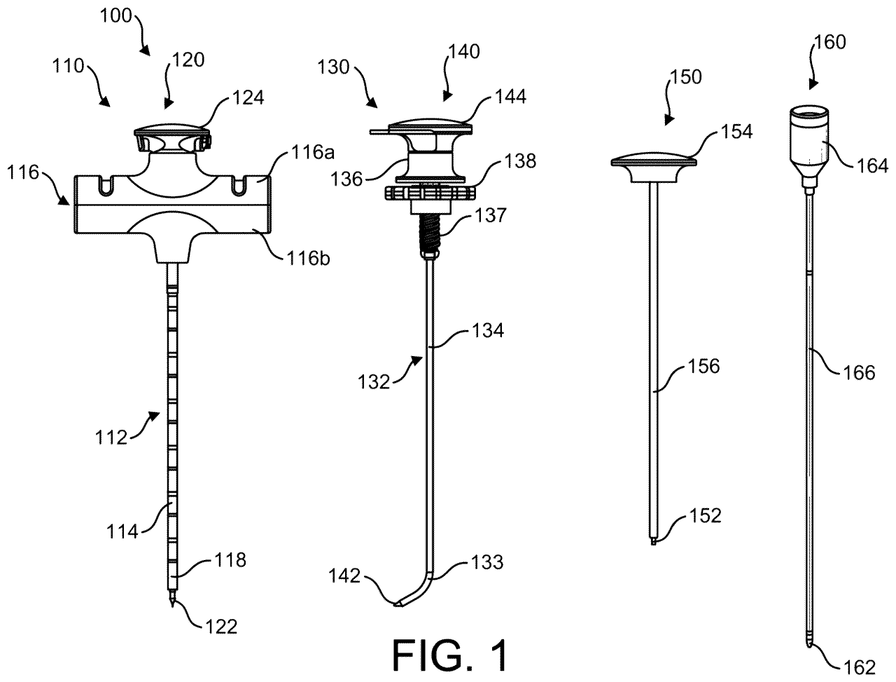

FIG. 1 shows an example embodiment of a set of access tools and treatment device 100 for performing a minimally invasive procedure to access a target treatment region within a vertebral body and to modulate (e.g., ablate, stimulate, denervate) the one or more nerves (e.g., basivertebral nerve) therein. As shown in FIG. 1, the access tool set or system 100 includes an introducer assembly 110 comprising an introducer cannula 112 and an introducer stylet 120, a curved cannula assembly 130 comprising a curved cannula 132 and a J-stylet 140, a straight stylet 150, and a radiofrequency (RF) energy delivery device 160, such as an RF probe. The access tools and treatment device 100 may be provided as a system or kit. In some embodiments, the kit of access tools and treatment device 100 may optionally include one or more additional introducer cannulas 112, curved cannulas 132, and/or an additional straight stylet 150 possibly having a different length from the first straight stylet 150. In some embodiments, the kit may include at least two of every access tool and at least two treatment devices. The optional additional access tools may be adapted to access one or more additional vertebrae during a treatment procedure.

As illustrated in FIG. 1, the introducer cannula 112 comprises a straight introducer tube 114 with a distal end 118 and an introducer handle 116 that is attached to the straight introducer tube 114. The straight introducer tube 114 can be made of a hard and stiff material (e.g., metal, hard plastic, ceramic, or composite material). The introducer handle 116 at the proximal end may comprise an upper handle portion 116a and a lower handle portion 116b, and can be made of a plastic material or another type of suitable material that is capable of withstanding impact from a mallet. The introducer stylet 120 comprises a straight rod or shaft (not shown) having a pointed tip 122 at the distal end and a first handle 124 at the proximal end attached to the straight rod. The straight rod or shaft (not shown) with the pointed tip 122 may be made of a hard and stiff material, e.g., metal, hard plastic or ceramic, and the first handle 124 may be made of a plastic material or a material that is capable of withstanding impact. The pointed tip 122 may be round tipped, bevel tipped or trocar tipped. The introducer handle 116 and the introducer cannula 112 together have an internal channel from end-to-end configured to receive the introducer stylet 120 such that the pointed tip 122 at the distal end of the introducer stylet 120 protrudes an open distal end 118 of the straight introducer tube 114 of the introducer cannula 112. As such, during the RF ablation procedure when the introducer cannula assembly is used to form a channel in a vertebral body, the pointed tip 122 penetrates the bone tissue and leading the introducer tube 114 to advance. As can be seen in FIG. 1, the pointed tip 122 (and the connected straight rod) of the introducer stylet 120 is slightly smaller than the internal channel of the straight tube 144 of the introducer cannula 112. As such, the introducer stylet 120 can be freely inserted into and removed from introducer cannula 112.

Regarding the curved cannula assembly 130, the curved cannula 132 comprises a straight tube section 134, a distal curved tube section 133 that is extended from the straight tube section 134, and a proximal end 136. The proximal end 136 comprises an adjustment wheel 138 threaded onto a thread section 137. By turning the adjustment wheel 138, the adjustment wheel 138 moves along the length of the thread section 137 of the proximal end 136 so that a distance between the adjustment wheel 138 and an end surface of the proximal end 136 can be adjusted. The curved distal tube section 133 of the curved cannula 132 comprises an elastic or resilient material that can be bent or straightened and can restore its original pre-curved shape.

The outside diameter of the distal curved tube section 133 and the elongate straight tube section 134 are made slightly smaller than the diameter of the internal channel of the straight introducer tube 114 of the introducer cannula 112. In this way, the curved cannula assembly 130 can be inserted into and removed from the introducer tube 114. The diameter matching between the distal curved tube section 133 and the internal channel of the introducer cannula 112 is similar to the diameter matching between the introducer stylet 120 and the introducer cannula 112. As such, the resilient curved distal curved tube section 133 can be straightened and inserted into the internal channel of the introducer cannula 112. The total length of the distal curved tube section 133 and the straight tube section 134 of the curved cannula 132 is configured such that, when fully engaged, the distal curved tube section 133 of the curved cannula 132 can at least partially penetrate through the straight introducer tube 114 of the introducer cannula 112 and come out of the open distal end 118 of the introducer tube 114. Once out of the open distal end 118 of the straight introducer tube 114, the distal curved tube section 133 of the curved cannula 132 will recover its curved shape because it is made of a resilient material (e.g., shape memory material).

When fully engaged, the adjustment wheel 138 at the proximal end 136 of the curved cannula 132 is in contact with an upper end surface of the introducer handle 116 of the introducer cannula 112. The length of the curved tube section 133 coming out of the straight introducer tube 114 of the introducer cannula 112 depends on the location of the adjustment wheel 138 on the thread section 137 of the proximal end 136. When the adjustment wheel 138 is moved to the lowest point of thread section 137, the length of the distal curved tube section 133 protruding out of the open distal end 118 of the straight introducer tube 114 is the shortest. On the other hand, when the adjustment wheel 138 is at the highest point of thread section 137, as illustrated in FIG. 1, the length of the distal curved tube section 133 protruding out of the open distal end 118 of the straight introducer tube 114 is the longest. During an RF ablation procedure, when a channel is formed in a vertebral body, the distal curved tube section 133 helps penetrate the bone tissue medially, toward the center area of the vertebral body. The length of the medial path is controlled by the adjustment wheel 138 and the malleting of the curved cannula assembly.

In FIG. 1, the J-stylet 140 comprises a rod portion (not shown) with a distal tip 142 and a second handle 144 attached to the rod portion (not shown). The distal section of the rod portion (not shown) of the J-stylet 140 is curved having the same or similar curvature of the distal curved tube section 133 of the curved cannula 132, and comprises an elastic or resilient material that is capable of bending and restoring its original shape. The curved cannula 132 includes an internal channel running through its length from the proximal end 136 through the tube sections 134 and 133 and to an open distal end at the curved tube section 133. The rod portion (not shown) of the J-stylet 140 is slightly smaller than the internal channel formed in the curved cannula 132. As such, the J-stylet 140 is configured to be received in the internal channel in the curved cannula 132 such that the distal tip 142 of the J-stylet 140 slightly protrudes from the open distal end of the curved cannula 132. When the J-stylet 140 is fully engaged with the curved cannula 132, the second handle 144 of the J-stylet 140 is in contact with proximal end 136 of the curved cannula 132, and the distal tip 142 of the J-stylet 140 is slightly outside of the open distal end of the curved tube section 133. The distal tip 142 can comprise a hard and stiff material, e.g., metal, and can be tipped, e.g., round tipped, bevel tipped or trocar tipped, so that it can help the advancement of the curved tube section 133 of the curved cannula 132 within a bone. The curved cannula 132 and the J-stylet 140 may each comprise a straight proximal main section and a curved distal section. The location and curvature of the curved distal sections of the curved cannula 132 and the J-stylet 140 may correspond to each other. The proximal end 136 of the curved cannula 132 and second handle 144 of the J-stylet 140 may each comprise markings or features to indicate the direction of the curved sections so that the curved sections can correspondingly align to each other and the curved tube section 133 of the curved cannula 132 and the distal tip 142 of the J-stylet 140 can be steered together during a procedure.

In FIG. 1, the straight stylet 150 comprises a straight rod 156 with a distal tip 152 and a third handle 154 attached to the straight rod 156 at the proximal end. At least the distal portion of the straight rod 152 comprises an elastic or resilient material that is capable of bending and restoring its original shape when bending force is relieved. Further, the outer diameter of the straight rod 156 of the straight stylet 150 may be similar to that of the rod portion of the J-stylet 140. As such, the rod 156 can fit into the internal channel of the curved cannula 132 like the J-stylet 140. When fully engaged, the distal tip 152 of the straight stylet 150 penetrates out of the open distal end of the distal curved tube section 133 of the curved cannula 132. Once out of the open distal end of the curved cannula 132, the distal section of the straight stylet 150 recovers its straight or another pre-formed shape. The curved cannula 132, the J-stylet 140 and the straight stylet 150 are constructed in such a way that the J-stylet 140 and the straight stylet 150 can be inserted into and removed from the internal channel of the curved cannula 132 during an ablation procedure. The construction consideration may include diameter matches between the internal channel of the curved cannula 132 and the rod portions of the J-stylet 140 and the straight stylet 150. The distal tip 152 can comprise a hard and stiff material, e.g., metal, and can be tipped, e.g., round tipped, bullet shaped, bevel tipped or trocar tipped. The J-stylet 140 and the straight stylet 150 may either be made of a radiopaque material or include an embedded radiopaque marker band at the distal tip to facilitate visualization under fluoroscopic or CT imaging.

The various proximal end features, including the introducer handle 116, the first handle 124, the proximal end 136, second handle 144, and the third handle 154 can be made of similar materials and be attached to their corresponding tube or rod sections similarly. For example, the introducer handle 116 can be made of a material that can withstand impact, e.g., plastic, wood, aluminum alloy, or composite material, because when assembled the introducer assembly 110 may be configured to receive impact from a mallet in order for the pointed tip 122 and the distal end 118 of the straight introducer tube 114 to penetrate and advance into a body part, e.g., a pedicle of a vertebra. If the introducer handle 116 is made of plastic, it can be insert molded with the straight introducer tube 114, which may be made of metal, hard plastic, ceramic, or another suitable rigid material. Or the introducer 116 and introducer tube 114 can be 3D printed with polymeric, metal or alloy based materials. If the introducer handle 116 is made of wood, it can be machined and can be attached to the straight introducer tube 114 by adhesive or interference fit attachment. On the other hand, if the introducer handle 116 is made of an aluminum alloy, it can be made by die casting, and can be attached to the straight introducer tube 114 by adhesive, interference fit attachment, or by welding. The attachment of the introducer handle 116 to the straight introducer tube 114 can be configured to ensure no relative movement between the two parts once attached. The first handle 124, the proximal end 136, the second handle 144 and the third handle 154 can be made and be attached to their corresponding tube or rod sections similar to the introducer handle 116.

Portions of the access tools, including the curved cannula 132, the J-stylet 140, and the straight stylet 150 may be formed of a variety of elastic materials that can be deformed under force and restore their shapes when the deforming force is relieved. For example, the curved tube section 133 of the curved cannula 132 and the distal end portion of the J-stylet 140 need to be straightened when entering the internal channel of the introducer cannula 112, and should curve back to their original curved shape once coming out of the open distal end 118 of the straight introducer tube 114 of the introducer cannula 112. Further, when inserted into the internal channel of the curved cannula 132, the distal portion of the rod section 156 of the straight stylet 150 needs to bend along the curved tube section 133, and straighten back once coming out of the open distal end of the curved tube section 133. The flexibility and resilience of an access tool may depend on its dimensions, especially the cross-sectional dimensions, and the material it is made of. In some embodiments these flexible and resilient portions of the access tools can be made of one or more plastic materials, e.g., polyamide (PA), polyethylene terephthalate (PET), polycarbonate (PC), polyoxymethylene (POM), acrylonitrile butadiene styrene (ABS), polyethylene (PE), polypropylene (PP), and polyimide (PI). In some embodiments, when the cross-sectional dimensions are relatively thick, they can also be made of one or more of the hard plastic elastomers, e.g., polyethylene-based polyolefin elastomers, polypropylene-based elastomers, thermoplastic polyester elastomers, thermoplastic polyurethane elastomers, nitrile butadiene rubber, and thermoplastic vulcanizate polymers. For example, the distal portion of the straight rod 156 can be a solid rod made of a plastic elastomer having a high Shore durometer measurement. In some embodiments, if the resilient tool portions have small cross-sectional dimensions or tubular structure to facilitate bending, they can be made of a semi-rigid materials, because when cross-sectional dimensions are sufficiently small proper flexibility can be achieved. Further, chevron designs or patterns, or through-thickness slits or holes can be applied to make a rigid portion more flexible. For example, if the straight tube section 134 and the curved tube section 133 of the curved cannula 132 are made of a hard and rigid material, patterned through-thickness slits can be formed on the distal curved tubular structure to increase flexibility or bendability.

Also shown in FIG. 1 is the RF ablation device 160, comprising a connection port 164 and a rod section 166 attached to the connection port 164. The rod section 166 comprises a flexible outer wall, e.g., a flexible plastic tubing, housing wires inside to electrically connect the connection port 164 to electrodes at a distal end 162 of the RF ablation device 160. The RF ablation device 160 is configured to apply RF power or energy sufficient to cause ablation or other modulation of tissue (e.g., one or more nerves) for pain relief treatment. The rod section 166 of the RF ablation device 160 can be about the same outer diameter as the tube sections 133 and 134 of the J-stylet 140 and the rod 156 of the straight stylet 150, and can be received into the internal channel of the curved cannula 132.

The one or more access tools and treatment device (e.g., the RF ablation device 160) may include an indicator configured to alert a clinician a current operation state of the treatment device. For example, the indicator may include an embedded radiopaque marker ring disposed along a length of, and extending around a circumference of, the treatment device 160, e.g., within or close to the ablation probe 160 to facilitate visualization under fluoroscopic or CT imaging.

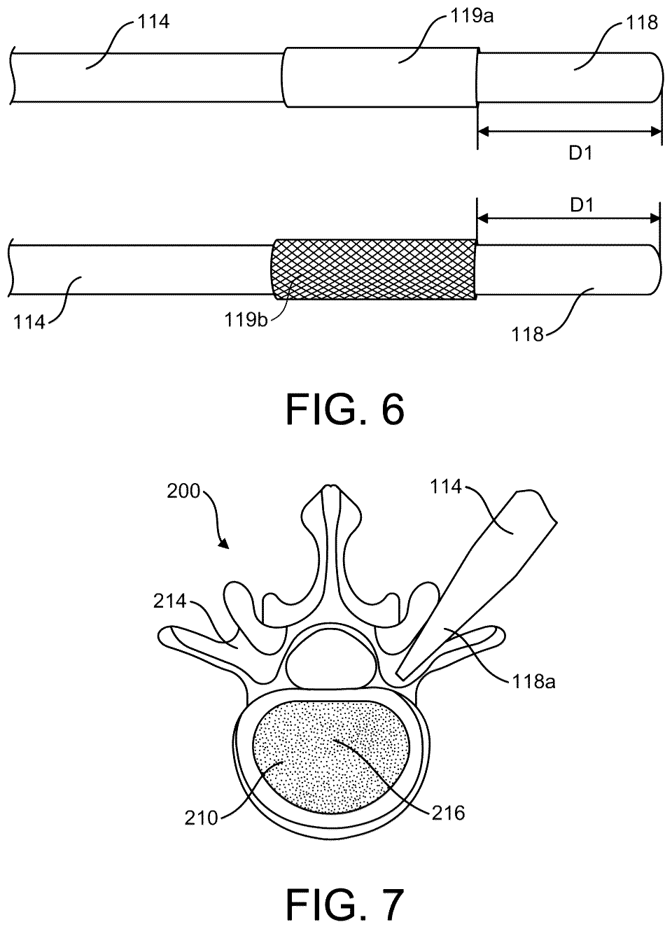

Applicant has provided and described examples of access tools and a treatment device in connection with FIG. 1 that may be used in connection with the various embodiments of introducer tools (e.g., introducer cannulas) or access techniques described herein in connection with FIGS. 6-24B to provide context but the embodiments can be used in connection with other access tools (e.g., introducers, cannulae, stylets) and treatment devices.

### B. Example Treatment Procedure

The tool set 100 as shown in FIG. 1 can be used, for example, to target a basivertebral nerve inside a vertebral body for the treatment of chronic low back pain. First of all, the subject patient with chronic low back pain can be examined to determine the specific vertebra or vertebrae that are the source of the chronic low back pain. In accordance with several implementations, target, or candidate, vertebrae for treatment, e.g., one or more of the lumbar vertebrae, sacral vertebrae, cervical vertebrae, or thoracic vertebrae, can be identified prior to treatment. Research has established that Modic changes and associated endplate damage strongly correlate with chronic low back pain. Therefore, one or more visualization modalities (e.g., magnetic resonance imaging (MRI), computer tomography (CT), X-ray, fluoroscopic imaging) may be used to determine whether a vertebral body or vertebral endplate exhibits active Modic characteristics or pre-Modic change characteristics (e.g., characteristics likely to result in Modic changes, such as Type 1 Modic changes, e.g., inflammation and edema, or type 2 Modic changes, e.g., changes in bone marrow and increased visceral fat content). For example, images obtained via MRI may be used to identify (e.g., via application of one or more filters) initial indications or precursors of edema or inflammation at a vertebral endplate prior to a formal characterization or diagnosis as a Type 1 Modic change. Accordingly, vertebral bodies may be identified as target candidates for treatment before Modic changes occur (or before painful symptoms manifest themselves to the patient) so that the patient can be proactively treated to prevent, or reduce the likelihood of, chronic low back pain before it occurs. In this manner, the patient will not have to suffer from debilitating lower back pain for a period of time prior to treatment.

In some implementations, a level of biomarker(s) (e.g., substance P, cytokines, or other compounds associated with inflammatory processes and/or pain) may be obtained from a patient (e.g., through a blood draw or through a sample of cerebrospinal fluid) to determine whether the patient is a candidate for basivertebral nerve ablation treatment. Cytokine biomarker samples may be obtained from multiple different discs or vertebral bodies or foramina of the patient and compared with each other in order to determine the vertebral bodies to target for treatment. Other biomarkers may be assessed as well. In some implementations, samples are obtained over a period of time and compared to determine changes in levels over time. For example, biomarkers may be measured weekly, bi-monthly, monthly, every 3 months, or every 6 months for a period of time and compared to analyze trends or changes over time. If significant changes are noted between the biomarker levels (e.g., changes indicative of pre-Modic, or Modic, changes as described above), treatment may be recommended and performed to prevent or treat back pain. Biomarker levels (e.g., substance P or cytokine protein levels) may be measured using various in vivo or in vitro kits, systems, and techniques (e.g., radio-immunoassay kits/methods, enzyme-linked immunosorbent assay kits, immunohistochemistry techniques, array-based systems, bioassay kits, in vivo injection of an anticytokine immunoglobulin, multiplexed fluorescent microsphere immune-assays, homogeneous time-resolved fluorescence assays, bead-based techniques, interferometers, flow cytometry, etc.). Cytokine proteins may be measured directly or indirectly, such as by measuring mRNA transcripts.

In some implementations, a target treatment region within a vertebral body may be clarified and pre-selected using pre-operative visualization (e.g., using bilateral fluoroscopy images or both anterior-posterior and lateral fluoroscopy images) of the vertebral body. The target treatment region may be identified as where a tip of a channeling stylet transects a basivertebral foramen (based on the images). The target treatment region may be a location or region between 10% and 50% of the distance between a posterior boundary or wall of the vertebral body and an anterior boundary or wall of the vertebral body. The vertebral body may be of a lumbar vertebra, a sacral vertebra, a thoracic vertebra, or a cervical vertebra. Multiple vertebral bodies may be treated. Nerves within other bones may also be treated.

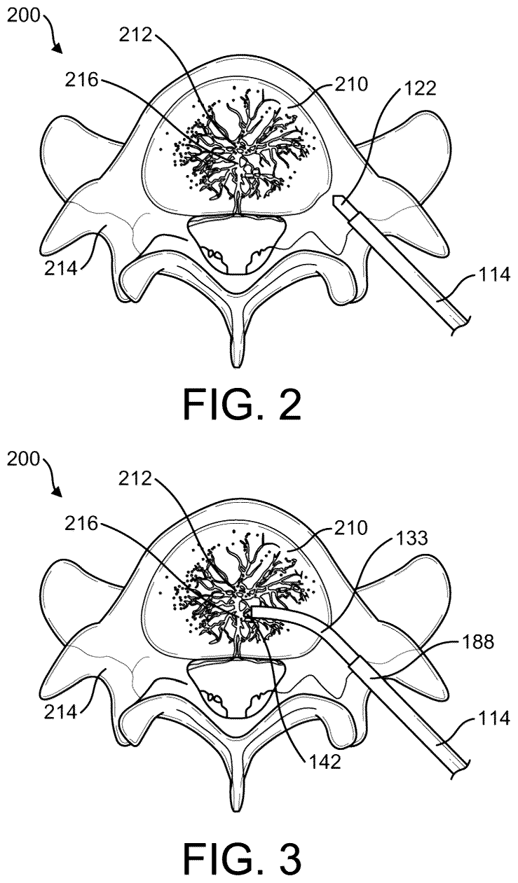

The treatment procedure may be monitored by an intraoperative visualization, e.g., fluoroscopic imaging, ultrasonic imaging, CT imaging, or MRI imaging, to track access and/or procedural progress. A small incision is made on the lower back of a subject patient to be treated. As shown in FIG. 2, the incision may be located on top of a pedicle 214 of a target vertebra 200, which has a vertebral body 210 having a basivertebral nerve 212 therein. A target treatment region 216 is usually pre-identified near the center of the vertebral body 210, where the basivertebral nerve branches to a plurality of axon terminals to innerve the superior and inferior endplates. In some implementations, the target treatment region 216 is in a posterior half of the vertebral body. In some implementations, the target treatment region 216 is between 10% and 60% (e.g., between 10% and 40%, between 30% and 50%) of the distance between the posterior wall and the anterior wall of the vertebral body. The introducer assembly 110 as shown in and described with FIG. 1, including the introducer cannula 112 and the introducer stylet 120 assembled together, is inserted into the incision. As such, when impact is received on the first handle 124 from a mallet, the sharp distal tip 122 of the introducer stylet 120 penetrates into the pedicle 214, leading the straight introducer tube 114 of the introducer cannula 112 to advance into the pedicle 214, forming a foramen, path, or channel. When it is determined from the monitoring imaging, e.g., fluoroscopic imaging, that the distal end 118 of the straight introducer tube 114 of the introducer cannula 112 has reached a predetermined location (e.g., an outer boundary of a vertebral body or a border of cortical bone and cancellous bone of the vertebral body), the introducer stylet 120 is removed from the internal channel of the introducer cannula 112, and the curved cannula assembly 130, including the curved cannula 132 and the J-stylet 140 assembled together, is inserted into the internal channel of the introducer cannula 112.

Referring to FIG. 3, as the curved section 133 of the curved cannula 132 penetrates out of the open distal end 118 of the straight introducer tube 114, it transitions to a curved configuration (e.g., based on its pre-curved shape memory properties or characteristics). Then the second handle 144 is malleted, further driving the distal curved tube section 133 of the curved cannula 132 to advance forward along a curved medial path, aiming at the target treatment region 216. The sharp distal tip 142 of the rod section of the J-stylet 140 penetrates slightly out of the open distal end of the distal curved tube section 133 of the curved cannula 132, cutting through the bone tissue and causing the open distal end of the distal curved tube section 133 to advance along when the second handle 144 is malleted. The direction the distal curved tube section 133 is pointing to can be steered or radially adjusted by turning the second handle 144. The advancement stops when the monitoring imaging shows that the open distal end of the distal curved tube section 133 has reached or almost reached the target treatment region 216, as shown in FIG. 3. To this point, the projected path or channel is formed through the pedicle 214 and into the vertebral body 210 by the access tools, e.g., the introducer cannula assembly 110 and the curved cannula assembly 130.

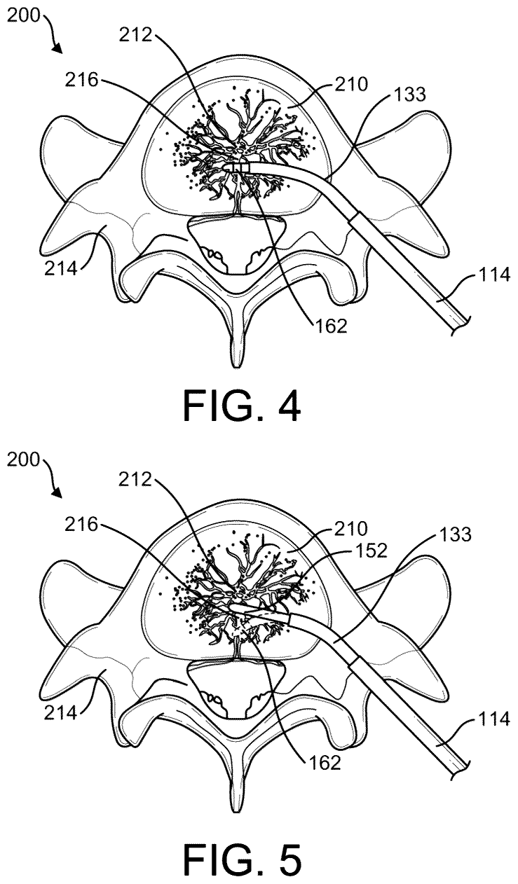

In FIG. 4, the J-stylet 140 is removed from the internal channel of the curved cannula 132, and the flexible RF ablation device 160 is inserted into the internal channel of the curved cannula 132. When the distal end 162 of the flexible RF ablation device 160 comes out of the open distal end of the curved tube section 133, it is positioned at the target treatment region 216. At this point, RF ablation can be performed. The distal end 162 comprises a bipolar electrode pair to generate radiofrequency (RF) energy that can cause temperature in the cancellous bone tissue around the target treatment region 216 to rapidly rise. The thermal energy may be conducted by heat transfer to the surrounding cancellous bone tissue, thereby heating up the cancellous bone portion. In accordance with several implementations, the thermal energy may be applied within a specific frequency range and having a sufficiently high temperature and over a sufficient duration of time to heat the cancellous bone such that the basivertebral nerve extending through the cancellous bone of the vertebral body is ablated or modulated. In several implementations, the modulation comprises permanent ablation or denervation or cellular poration, e.g., electroporation. In some implementations, the modulation comprises temporary denervation or inhibition. In some implementations, the modulation comprises stimulation or denervation without necrosis of tissue.

For the RF ablation, temperature of the cancellous bone portion surrounding the RF probe 162 (e.g., the target treatment region) may rise to a range from about 70 degrees Celsius to about 115 degrees Celsius (e.g., between 70 degrees Celsius and 85 degrees Celsius, between 75 degrees Celsius and 90 degrees Celsius, between 70 degrees Celsius and 80 degrees Celsius, between 75 degrees Celsius and 85 degrees Celsius, between 80 degrees Celsius and 100 degrees Celsius, between 90 degrees Celsius and 115 degrees Celsius, overlapping ranges thereof, or any value within the recited ranges). The temperature ramp rate may range from 0.1 degrees Celsius/second-5 degrees Celsius/second. The time of treatment may range from about 10 seconds to about 1 hour (e.g., from 10 seconds to 2 minutes, from 30 seconds to 90 seconds, from 1 minute to 5 minutes, from 2 minutes to 8 minutes, from 5 minutes to 15 minutes, from 10 minutes to 20 minutes, from 15 minutes to 30 minutes, from 30 minutes to 1 hour, overlapping ranges thereof, or any value within the recited ranges). Pulsed energy (e.g., pulsed RF) may be delivered as an alternative to or in sequence with continuous RF energy. For applying radiofrequency energy, the frequency applied may range from 350 kHz to 650 kHz. A power of the radiofrequency energy may range from 5 W to 30 W. In accordance with several implementations, a thermal treatment dose (e.g., using a cumulative equivalent minutes (CEM) 43 degrees Celsius model) is between 200 and 300 CEM.

As shown in FIG. 3, as the distal end, including the distal tip 142 of the J-stylet 140, of the curved assembly 130 advances through the pedicle 214 and the cancellous bone issue of the vertebral body 210, the monitoring imaging (e.g., intraoperative fluoroscopy) may indicate that the distal end of the curved assembly 130 will miss the target treatment region 216 (e.g., the desired trajectory needed to reach the target treatment region 216 will be off). For example, the projected path or trajectory of the distal end of the distal curved tube section 133 may be below (e.g., too posterior of) the target treatment region 216, or undershooting, as shown in FIG. 5 by the broken lines. This undershooting may be due to, for example, the penetration direction of the straight introducer tube 114 in to the pedicle 214 being slightly off, the penetration distance of the straight hypotube 114 into the pedicle 214 being not deep enough, and/or the curvature of the distal curved tube section 133 being slight sharper than projected (e.g., due to low density of osteopenic or osteoporotic bone). In this case, the advancement of the curved cannula assembly 130 may be ended or stopped prior to full advancement or may be retracted a short distance and the J-stylet 140 may be removed from the internal channel of the curved cannula 132. In some implementations, the straight stylet 150 is then inserted into the internal channel of the curved cannula 132. When the distal tip 152 of the straight stylet 150 penetrates through the open distal end of the curved cannula 132, it straightens out and advances forward in a straight path rather than following the curved trajectory of the distal curved tube section 133. Since no further curvature is added to the distance between the open distal end of the distal curved tube section 133 and the target treatment region 216, the distal tip 152 of the straight stylet may arrive at the target treatment region 216, correcting the undershooting issue, as shown in FIG. 5. At this point, the straight stylet 150 is removed from the curved cannula 132, and the RF ablation device 160 is inserted for ablation treatment.

Again referring back to FIG. 3, when curved cannula assembly 130 is inserted into the internal channel of the introducer cannula 112 and the second handle 144 is malleted to advance the curved cannula assembly 130 forward, there may exist friction between the tube sections 133, 134 and the internal channel of the introducer cannula 112. This friction force may cause advancement of the distal end 118 of the introducer tube 114 forward. In some instances, the advancement of the distal end 118 of the introducer tube 114 may cause the trajectory path of the distal end of the curved assembly 130 to miss the target treatment region 216 by overshooting. For example, the resulting projected path or trajectory of the distal end of the distal curved tube section 133 may be above (e.g., too anterior of) the target treatment region 216. Therefore, in some embodiments, it is advantageous to hold or anchor the distal end 118 of the introducer cannula 112 in place when the advancement of the distal end 118 of the introducer cannula 112 is stopped and the curved cannula assembly 130 or another device is inserted into the internal channel of the introducer cannula 112.

Applicant has provided and described examples of access tools and a treatment device in connection with FIGS. 2-5 that may be used in connection with the various embodiments of introducer tools or access techniques described herein in connection with FIGS. 6-24B to provide context but the embodiments can be used in connection with other procedures as well.

### C. Features on the Distal End of the Introducer Tube to Resist Movement

FIG. 6 presents example solutions to resist movement of the distal end portion 118 of the introducer cannula 112 when deployment of the introducer cannula 112 is complete. In FIG. 6, the introducer tube 114 shown in the top image includes a resistance portion or region 119a that is located adjacent or next to the distal end portion 118 of the introducer cannula 112 and is slightly larger than the distal end portion 118 of the introducer cannula 112, forming an edge or abutment surface at the transition between the distal end portion 118 and the resistance portion or region 119a. The resistance portion or region 119a may be formed by sleeving another larger tube portion outside the introducer tube 114, or by locally enlarging a portion of a tube, causing a swelling effect. The distance between the edge of the slightly larger portion 119a and a terminus or tip of the distal end portion 118 of the introducer cannula 112 is labeled as D1, as shown in FIG. 6. This distance D1, or the length of the distal end portion 118 of the introducer cannula 112, may be slightly smaller than or about the same as the projected or desired penetration depth of the distal end portion 118 into the pedicle 214. As such, when the projected or desired penetration depth of the distal end portion 118 is reached and the curved cannula assembly 130 or another device is inserted into the internal channel of the introducer cannula 112, the slightly larger dimension of the resistance portion 119a helps slow down or impede the distal end 118 of the introducer cannula 112 from further advancing into the pedicle 214 or the vertebral body 210. Thus, the resistance portion 119a acts as a depth stop or depth limiter.

The example embodiment shown in the bottom image of FIG. 6 has a slightly larger resistance portion 119b on the introducer tube 114 that is similar to the resistance portion 119a of the example embodiment of the top image. However, the resistance portion 119b further comprises knurled marks or patterns on the lateral surface. These knurled marks or patterns make the surface rough and further increase resistance to movement of the distal end portion 118 of the introducer cannula 112.

The distal end portion 118 of the introducer cannula 112 can be modified to have a geometric shape to achieve the purpose of slowing down, resisting, limiting or preventing the movement of the introducer cannula 112. Example embodiments of such modifications are shown in FIGS. 7-9. In FIG. 7, the introducer tube 114 is constructed to have a tapered distal end portion 118a, which has a constant taper in cross-sectional dimension (e.g., diameter), thereby forming a smaller tip than a base that is connected to the introducer tube 114 of the introducer cannula 112, which may have a uniform cross-sectional dimension (e.g., diameter). When the distal end portion 118a enters into the pedicle 214 following a projected path or trajectory, the progressively increasing cross-sectional dimension (e.g., diameter) of the distal end portion 118a creates progressively increasing resistance to slow down or impede further advancement of the distal end 118a of the introducer cannula 112 as it advances further within bone (e.g., through a pedicle). When the deployment of the introducer cannula 112 is complete and the curved cannula assembly 130 or another device is inserted into the internal channel of the introducer cannula 112, the frictional force between the curved cannula 132 or another device and the internal channel of the introducer cannula 112 may be sufficiently small compared to the increased resistance to advancement due to the tapered distal end portion 118a, such that the introducer cannula 112 does not further advance into the pedicle 214 and the vertebral body 210.

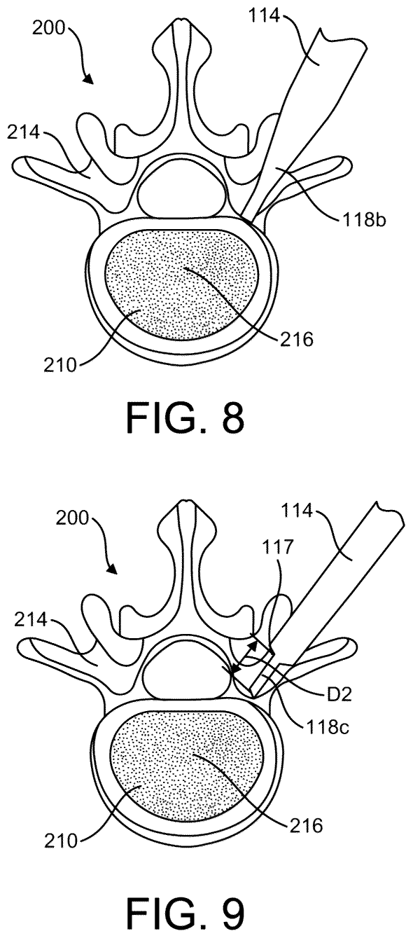

In FIG. 8, a distal end portion 118b of the introducer tube 114 has a constant or uniformly sized distal tip portion (e.g., having a constant or uniform cross-sectional dimension such as diameter) that is smaller than the size (e.g., cross-sectional dimension such as a diameter) of the introducer tube 114 and a tapered proximal portion disposed between the smaller distal tip portion and the introducer tube 114. The constant or uniformly sized smaller distal tip portion may have a predetermined length. After the smaller tip portion of the distal end portion 118b penetrates the pedicle 214, the tapered proximal portion starts to impede the advancement because of the increased cross-sectional dimension. When the introducer stylet 120 is withdrawn and the curved cannula assembly 130 or another device is inserted into the internal channel of the introducer cannula 112, the tapered proximal portion of the distal end portion 118b may help impede or stop further advancement of the distal end portion 118b of the introducer cannula 112.

Referring to FIG. 9, an example embodiment of the introducer tube 114 or introducer cannula 112 shows a distal end portion 118c having a step (or edge) 117 at the transition between a smaller distal tip portion (e.g., smaller in cross-sectional dimension such as a diameter) and the introducer tube 114 having a larger cross-sectional dimension. The smaller distal tip portion may have a constant thickness or a slightly tapered profile and a predetermined length, D2, as shown in FIG. 9. After the smaller distal tip portion penetrates the pedicle 214, the step 117 eventually engages the outer surface of the pedicle 214 and impedes or stops further advancement of the introducer tube 114 and thus, the introducer cannula 112. If the size of the step 117 is sufficiently large, the step 117 may totally or completely stop the advancement of the introducer tube 114 after the distal end portion 118c fully enters the pedicle 214.

### D. Adjustable Hypotube

The example embodiment shown in FIG. 9 may impede or stop the advancement of the distal end portion 118c and the introducer cannula 112 depending on the size of the step 117. Nevertheless, in accordance with some embodiments, since the smaller distal end portion 118c is pre-formed on the introducer tube 114 of the introducer cannula 112, the length D2 cannot be adjusted. This fixed penetration distance D2 determines the penetration depth of the introducer cannula 112 into the pedicle 214 and may impose limitations. For example, the lumbar vertebrae, the thoracic vertebrae, the cervical vertebrae, and sacral vertebrae may have anatomical structures, distances, and shapes that are different from each other, and may require different penetration depths from the outer surfaces of the pedicle to within the vertebral body in order to form an ideal target distance, path or trajectory to the target treatment region 216 in the vertebral body. Even within the lumbar section of the spine, the LI vertebra may be constructed differently from the L5 vertebra and may require a different penetration depth in the pedicle to reach the target treatment region 216 in the vertebral body. In addition, there may be differences based on age, gender, or other patient-specific characteristics (such as bone density, body fat, pre-existing surgical hardware, spinal deformities or abnormalities). The target treatment region 216 within a vertebral body 200 and the projected access path or channel, including the penetration depth of the distal end portion 118 of the introducer cannula 112 into the pedicle 214, may be pre-predetermined using pre-operative visualization (e.g., using bilateral fluoroscopy images or both anterior-posterior and lateral fluoroscopy images or oblique views) of the vertebra(e). Therefore, in accordance with several embodiments, it may be advantageous if the length of the distal end portion 118 can be adjustable to satisfy the different requirements of the particular patient or the particular vertebra or other bone).

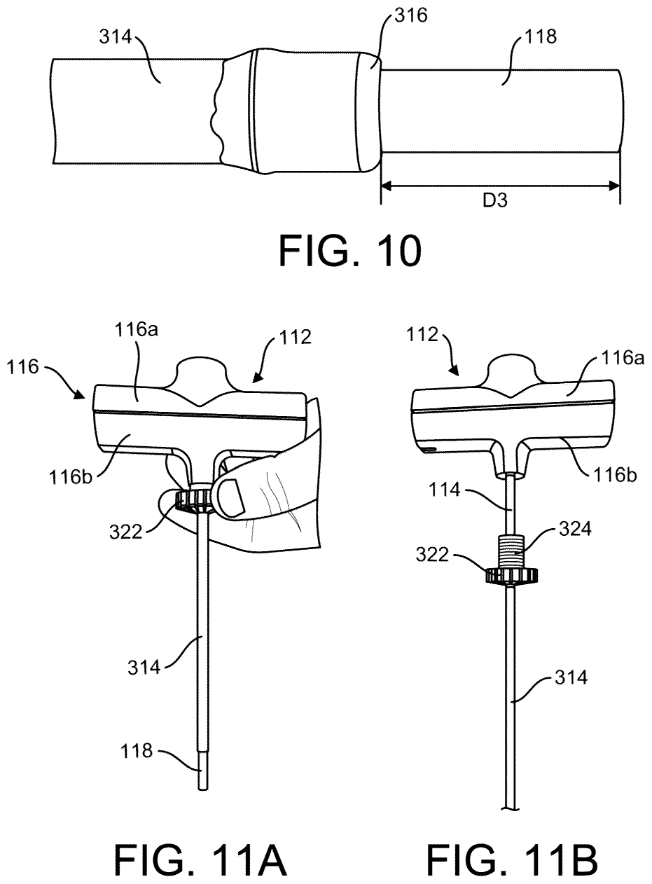

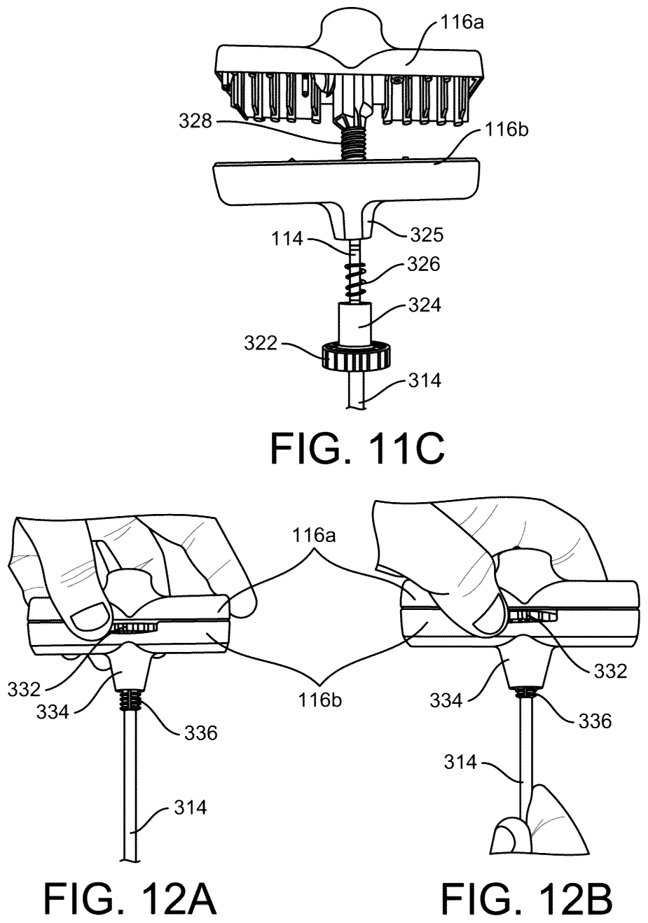

An example embodiment of an introducer cannula having an adjustable length for the distal end portion 118 is shown in FIG. 10, wherein the introducer cannula 112 comprises a second tube (e.g., hypotube) 314 concentrically surrounding a first tube (e.g., introducer tube) 114. The hypotube 314 extends from an introducer handle 116 at a proximal end to a hypotube end or step 316 at a distal end. As shown in FIG. 11A, the distal end portion 118 of the introducer cannula 112 extends out of the step 316 of the hypotube 314 forming a length D3 (shown in FIG. 10) from the step 316 to the terminus or tip of distal end portion 118. The inner diameter of the hypotube 314 is slightly larger than the outer diameter of the introducer tube 114, so that longitudinal movement between the hypotube 314 and the introducer tube 114 provides minimal, or reduced, resistance. As shown in FIG. 10, the step 316 has a larger cross-sectional dimension (e.g., diameter) than the distal end portion 118 of the introducer cannula 112, forming a step or edge or abutment surface to engage the outer surface of the pedicle 214 or other bone when the appropriate or desired extent of advancement of the introducer cannula 112 into the pedicle 214 is achieved. Therefore, the hypotube 314 disposed on introducer tube 114 allows the introducer tube 114 to extend out of the hypotube 314. As such the distal end length D3 is positive and is always larger than 0. When the increase in cross-sectional dimension formed by the step 316 is large enough, total stoppage or termination of further advancement can be achieved. As such, the distal end portion 118 of the introducer cannula 112 can be stopped or anchored in place when the curved cannula 132 or another device is inserted into the introducer cannula 112 to further form a channel into the vertebral body 200 to reach the target treatment region 216. The step 316 may have a blunt curvature (e.g., may be partially bullet shaped, beveled or wavy shaped) for friendly engagement with the outer surface of the pedicle 214. In some embodiments, the step 316 may have teeth, may be knurled, and/or may be media blasted to provide a rough surface for more resistance. The hypotube 314 with the shaped step 316 can be made of a stiff material, such as but not limited to, stainless steel, nitinol, doped plastic, polymeric or ceramic material.

The length D3 from the step 316 of the hypotube 314 to the distal tip of the introducer tube 114 can be made adjustable before or during a procedure (e.g., an RF ablation procedure or other diagnostic or treatment procedure) by adjusting the length of the hypotube 314 extending longitudinally from the proximal handle 116 of the introducer cannula 112. Various embodiments can be implemented. The following are a few examples.

FIGS. 11A-11C illustrate an example embodiment of an introducer cannula that includes a threaded coupling for adjusting the extension of the hypotube 314 in the longitudinal direction from the proximal end of the introducer cannula. As shown in FIG. 11A and FIG. 11B, an introducer cannula 112 comprises an upper handle portion 116a and a lower handle portion 116b coupled together to form an introducer handle 116. The introducer cannula 112 further comprises a hypotube 314 attached to a rotatable knob, nut or wheel feature 322 that has a tubular knob extension 324. The attachment between the knob, nut or wheel feature 322 and the hypotube 314 may be accomplished by insert molding, adhesive, 3D printing, or by interference fit (e.g., pressed fit or friction fit), so that longitudinal movement of the knob, nut or wheel feature 322 (e.g., via rotational movement) causes longitudinal movement of the hypotube 314 as well. The structure of the proximal introducer handle 116 is more clearly depicted in FIG. 11C, where the upper handle portion 116a and the lower handle portion 116b are separated to reveal certain internal structures. As shown in FIG. 11C, the upper handle portion 116a has a threaded extension 328 having an external thread and attached to or protruded from the introducer tube 114. The attachment may be achieved by insert molding, adhesive, 3D printing, and/or interference fit, for example. In some embodiments, the threaded extension 328 can be made as a part of the upper handle portion 116a. The knob extension 324 has a hollow structure at the proximal side and is affixed to the hypotube 314 at the distal side. The hollow structure of the knob extension 324 may hold a compression spring 326 between the wall of the knob extension 324 and the introducer tube 114. The inside wall of the knob extension 324 has an internal thread formed thereon and is configured to mate with the threaded extension 328 on the upper handle portion 116a. The lower handle portion 116b has a protruding boss 325 with a hole formed therethrough configured to accept the compression spring 326 and the knob extension 324.

When the upper handle portion 116a and the lower handle portion 116b are assembled, the knob extension 324 is inserted in the hole of the protruding boss 325 of the lower handle portion 116b. By rotating the knob 322, the internal thread on the knob extension 324 is mated with the external thread on the threaded extension 328 connected to the upper handle portion 116a. Further rotation of the knob 322 moves the knob 322 longitudinally toward the proximal end of the introducer cannula 112. Turning the knob 322 in the reverse direction moves the knob 322 toward the distal end of the introducer cannula 112. In this way, the hypotube 314 can be moved longitudinally toward the proximal end or toward the distal end by turning the knob 322, effectively adjusting the length D3 between the hypotube step 316 and the tip of the distal end portion 118. Of course, the operation could be opposite in some embodiments. During the adjustment, the compression spring 326 is sandwiched between a base of the hollow structure in the knob extension 324 and the tip of the threaded extension 328, thereby biasing the hypotube 314 toward the distal direction. The biasing force causes a friction force between the internal thread in the knob extension 324 and the external thread on the threaded extension 328, thereby providing positioning stability when the longitudinal positioning adjustment of the hypotube 314 is completed.

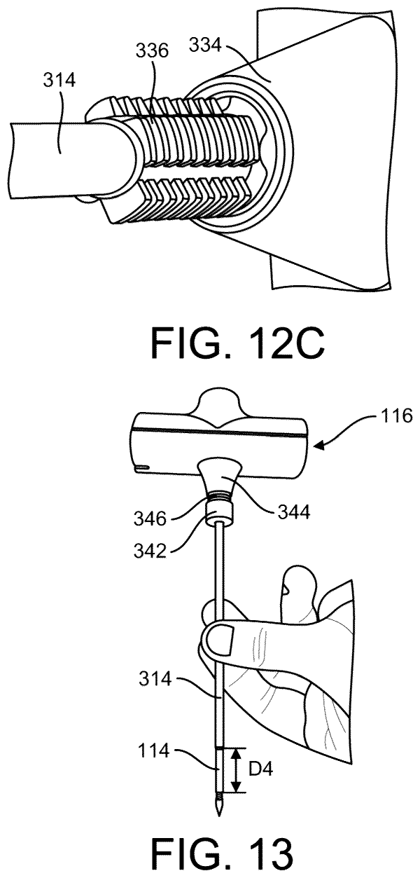

Another example embodiment of threaded coupling is shown in FIGS. 12A-12C. Between the upper handle portion 316a and the lower handle portion 316b is sandwiched a wheel 332 that is coupled with the hypotube 314. The coupling between the wheel 332 and the hypotube 314 allows the wheel 332 to transfer rotational movement to the hypotube 314, and at the same time allows the hypotube 314 to freely move longitudinally. For example, the wheel 332 can have a square (or another polygon shaped) opening at the center and the proximal end (not shown) of the hypotube 314 may have a matching outer shape to mate with the opening in the wheel 332. As shown in FIG. 12C, a threaded portion 336 having an external thread formed thereon is integrated with the hypotube 314. The threaded portion 336 may comprise a plastic material that is insert molded with the hypotube 314. An alternative method of integration may be accomplished by 3D printing. A protruded portion (e.g., a boss) 334 has a through hole formed in the middle with an internal thread formed therein configured to mate with an external thread of the threaded portion 336. When assembled, turning the wheel 332 sandwiched in the introducer handle 116 moves the threaded portion 336 longitudinally toward distal end, as shown in FIG. 12A, or toward the proximal end, as shown in FIG. 12B, effectively changing the length of the distal end portion 118. The wheel 332 could be substituted with a knob, switch, slider, or other toggle or adjustment mechanism.

In some embodiments, the threaded coupling embodiments of FIGS. 11A-11C and FIGS. 12A-12C may include anti-backlash mechanisms or systems (such as but not limited to ball detents pushing into grooves) so as not to allow the wheel 332 or other adjustment mechanism to move (e.g., turn or rotate) in an opposite direction. Other anti-backlash mechanisms or systems may include one-way bearings that allow movement only in one direction (e.g., distally), spring-loaded inserts that maintain tension so as not to allow unwinding, and/or spring-loaded gear teeth that allow for one-way movement that could be separated to allow for reuse.

In FIG. 13 is shown an embodiment of a Tuohy-Borst style system for adjusting the distance D4 of the distal end portion 118. The Tuohy-Borst style system may be implemented in a handle or may be free floating as an attachment similar to a shaft collar on a rod. In some embodiments, a rotatable or turning wheel knob 342 having internal threading is mated on an external thread portion 346 formed at the end of a protruding boss 344 of the lower handle portion 116b. Inside the knob 342 and between the threaded portion 346 and the hypotube 314 is disposed a rubber ring (not shown) that is resilient and deformable under force. As shown in FIG. 13, the hypotube 314 can be moved proximally or distally by one or more fingers until a desired length D4 is achieved. Then the knob 342 is turned to move the knob 342 toward the proximal direction. As such, the rubber ring (not shown) is squeezed between the knob 342 and the tip of the threaded portion 346 of the protruding boss 344. The squeezed rubber ring (not shown) presses against the hypotube 314 and effectively resists or stops further movement of the hypotube 314.

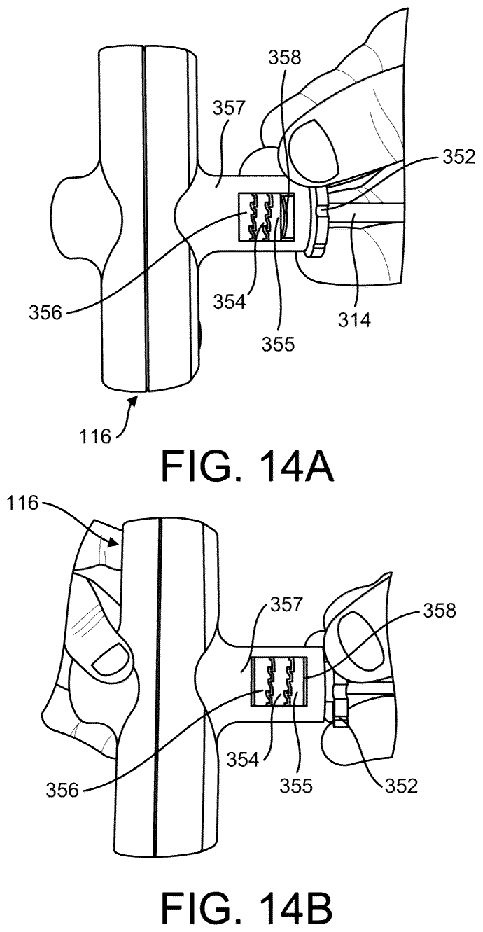

An example of a one-way gear system is presented in FIGS. 14A-14C. A rotatable wheel 352 is threadably coupled to an internal channel of a protruded boss 357. Inside the internal channel of the protruded boss 357 are stacked a one-way gear 354 sandwiched between a first gear 355 at the distal side and a second gear 356 at the proximal side. The three gears 355, 354 and 356 are pressed together by a bias compression spring 358 that is sandwiched between the first gear 355 and the wheel 352. The middle one-way gear 354 and the wheel 352 are attached to the hypotube 314 (e.g., by insert molding, adhesive, or any other suitable method). In some embodiments, turning the wheel 352 brings the one-way gear 354 and the hypotube 314 to turn also. The teeth of the one-way gear 354 are shaped like saw teeth, forming sharp corners of teeth toward one rotational direction. The teeth on both sides of the one-way gear 354 mirror each other. Each of the first gear 355 and the second gear 356 has teeth at the side facing the one-way gear 354. The shape of the teeth on the first gear 345 and on the second gear 356 match the shape of the teeth on the one-way gear 354 at the engagement side. In FIG. 14A, the wheel 352 is rotated to a circumferential position so that the teeth of the one-way gear 354 are fully in contact with the teeth on the first gear 355 and the teeth on the second gear 356, forming minimal gaps between the gears. Thus, the bias compression spring 358 ensures that the hypotube 314 is retracted closest to the proximal end. In FIG. 14B, the turning wheel 352 is turned to a circumferential position that gaps between the one-way gear 354 the neighboring first gear 355 and the second gear 366 are the largest. As such, the hypotube 314 is extended toward the distal end 118 the most. The adjustment of the hypotube 314 in the longitudinal direction depends on the teeth height. A mechanism may be implemented to stop the turning of the wheel 352 and therefore the length of the hypotube 314 after adjustment is held unchanging.

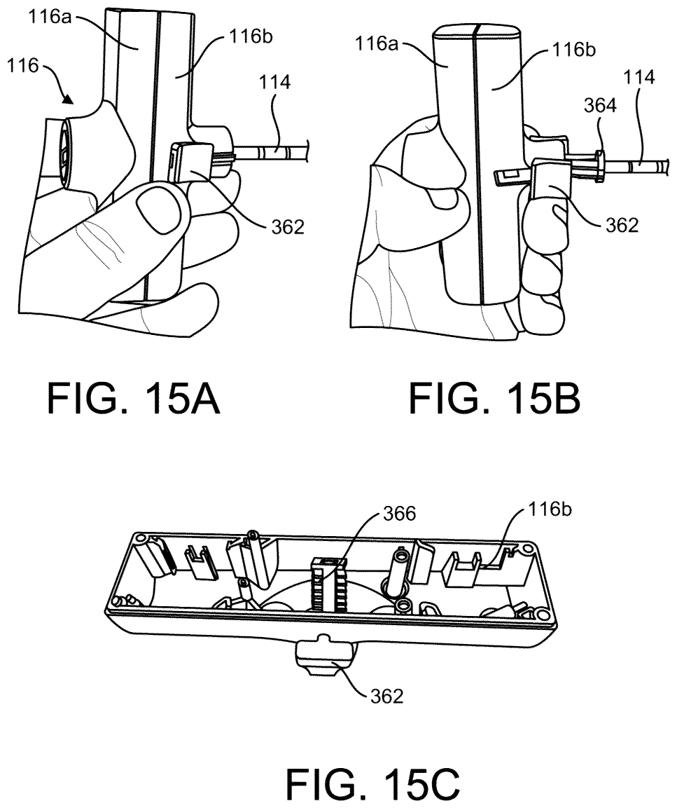

An example embodiment of a linear rack or ratchet type adjustment system is shown in FIGS. 15A-15C. In FIG. 15A, a first toggle control element 362 is shown coupled to the lower handle portion 116b in a retracted position. A second toggle control element 362 may be disposed at the other side of the lower handle portion 116b, forming a pair of toggle control elements 362 that can be activated by an operator (e.g., a thumb and a finger). In FIG. 15B, the toggle control element 362 is positioned more distally compared to the position in FIG. 15A. The toggle control element 362 as shown in FIG. 15B is connected to a ring 364 which is normally integrated with the hypotube 314 (not shown in FIG. 15A and FIG. 15B). As such, moving the toggle control element 362 between the retracted position and the extended position brings the hypotube 314 along when the hypotube 314 is integrated with the ring 364.

As shown in FIG. 15C, a gear rack 366 is disposed inside of the lower handle portion 116b. The gear rack 366 may be formed as part of the lower handle portion 116b (e.g., molded as one piece) or they may be assembled after they are made separately. The gear rack 366 may have gear teeth on both sides, and each of the toggle control elements 362 has at least one tooth to engage with the teeth of the gear rack 366. The slender connection between the toggle control element 362 and the ring 364 gives a weak spring force for the tooth on the toggle control element 362 to engage with the teeth of the gear rack 366. Meanwhile, the weak spring force allows the toggle control element 362 to be pushed by one or more fingers to move distally and proximally between the retracted and extended positions. As such, the hypotube 314, which is integrated with the ring 364, is retracted or extended longitudinally.

### E. Additional Controlled Bone Access Instrument Embodiments

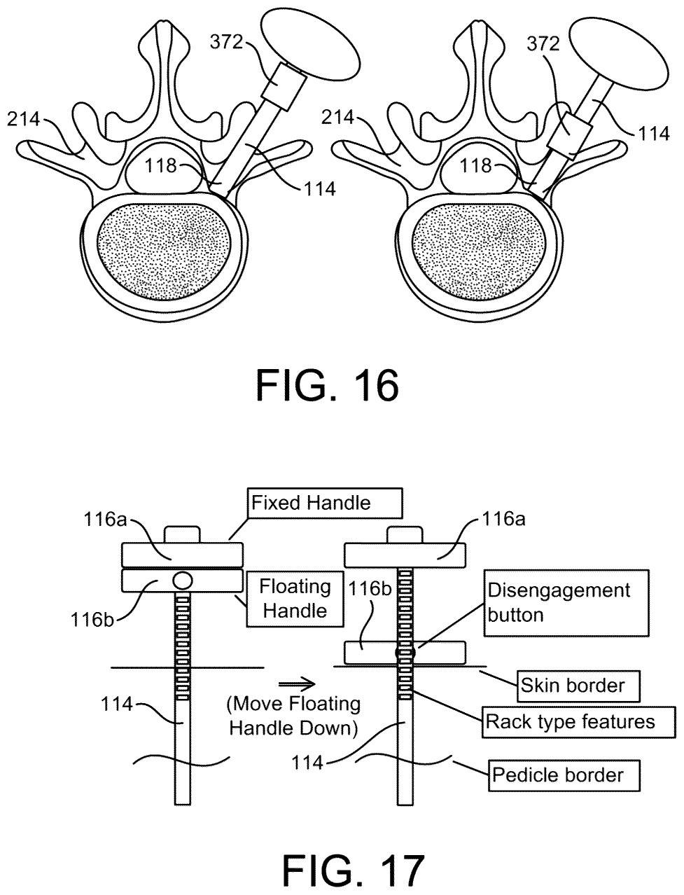

In FIG. 16, an embodiment comprises an adjustable block 372 coupled to the introducer tube 114 of the introducer cannula 112. The block 372 can embrace or surround the introducer tube 114 or can be attached to a partial circumferential outer surface of the introducer tube 114. After the distal end portion 118 of the introducer tube 114 advances into the pedicle 214 for a predetermined distance, the deployment of the introducer cannula 112 may be complete. The block 372 may then be moved to rest against the outer surface of the pedicle 214 and may be affixed in place (e.g., by a set screw or a pin). As such, further advancement of the introducer tube 114 may be prevented. The block 372 can also be made larger to rest against the skin surface on top of the vertebra of a patient.

Another embodiment is shown in FIG. 17, which includes a fixed handle portion 176a and a lower floating handle portion 176b. The floating handle portion 176b is constructed and configured to be movable longitudinally along the introducer tube 114 of the introducer cannula 112. For example, the floating handle portion 176b can include a toggle control element (e.g., disengagement button) 177 to cause an engagement element (not shown) within the floating handle portion 176b to disengage from a rack feature or element 178 along a proximal portion of the introducer tube 114 so that the floating handle portion 176b can be moved proximally or distally along the introducer tube 114. The toggle control element 177 can be released to cause the engagement element to reengage a rack feature or element 178 at the new longitudinal position. After the distal end portion 118 of the introducer cannula 112 advances in the pedicle 214 for a predetermined distance, the lower floating handle portion 116b is moved distally to be in contact with the skin surface to stop further advancement of the introducer tube 114, and thus introducer cannula 112.

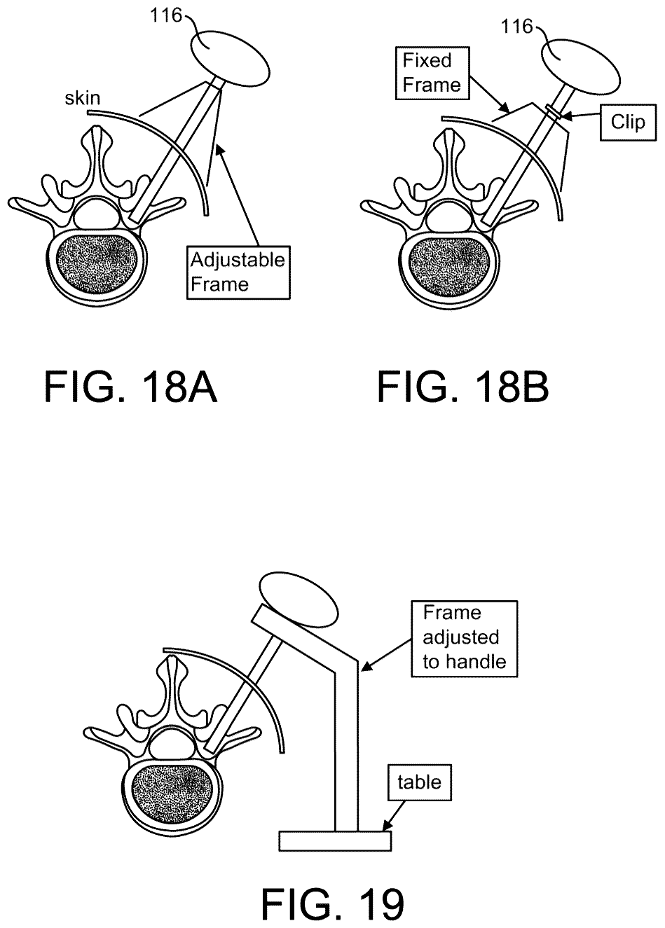

In FIG. 18, the introducer cannula 112 is supported by an external frame that rests on the skin surface and is disposed below the introducer cannula handle 116. In FIG. 18(a), the external frame is adjustable. After the distal end portion 118 of the introducer cannula 112 advances a predetermined distance into the pedicle 214, the frame is adjusted to give firm support to the introducer handle 116 so that further advancement of the introducer cannula 112 is prevented. In FIG. 18(b), the frame is fixed and not adjustable. After the distal end 118 of the introducer cannula 112 advances a predetermined distance into the pedicle 214, the frame is affixed to the introducer tube 114 (e.g., by one or more clips or set screws), to support the introducer cannula 112 so that further advancement of the introducer cannula 112 is prevented. FIG. 19 shows an example of another supporting frame. However, in this embodiment, the external frame rests on a procedure room table to perform the same function to support the introducer cannula 112. The external frame of FIG. 19 may be adjustable to provide controlled or guided pedicle access along a particular desired angle or trajectory. The external frames shown in FIG. 18 and FIG. 19 which rest either on the patient skin or on a table normally have a large contact area or no contact area. As such, local tissue damage, such as bruising, to the bone or skin can be prevented.

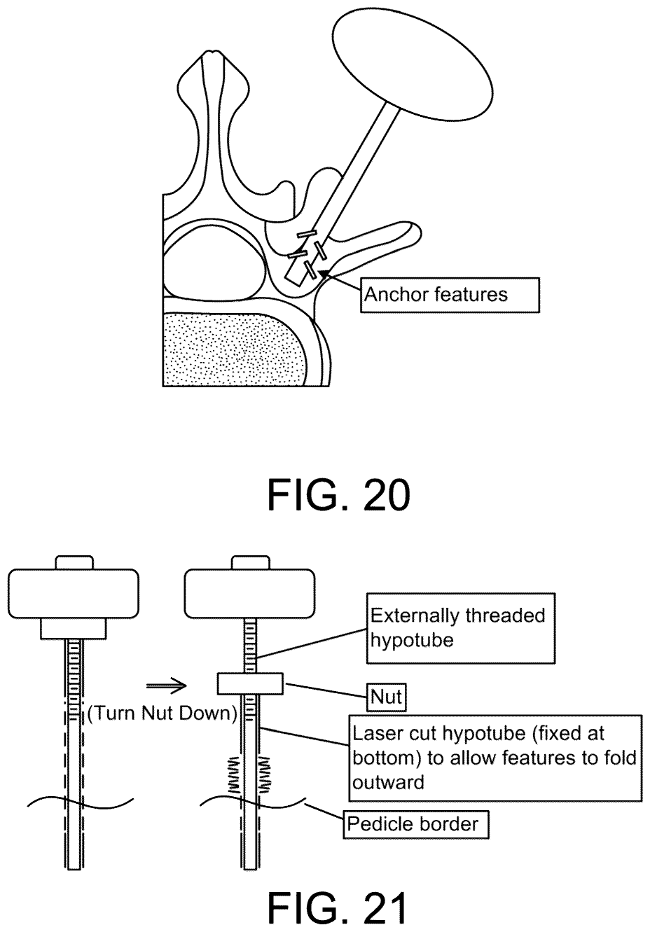

In FIG. 20, an example embodiment is shown in which anchor features positioned along the distal end portion 118 of the introducer cannula 112 prevent or resist further advancement of the introducer cannula 112. When the introducer cannula 112 advances into the pedicle 214, the anchor features expand outwardly to create resistance to impede the advancement. When the distal end 118 of the introducer cannula 112 is retracted or withdrawn from the formed channel in the pedicle 214, the anchor features collapse and the resistance to introducer cannula's movement is reduced.

FIG. 21 illustrates an example embodiment of an expanding sleeve with bellows features over the introducer tube 114 of the introducer cannula 112. When the introducer cannula 112 deployment is complete, the sleeve wrapped over the introducer tube is pushed downward by a nut, knob or wheel, so that the sleeve folds to bellows resting onto the outer surface of the pedicle 214. The expanding sleeve is then fixed relative to the introducer cannula 112 to prevent, resist or limit further advancement of the introducer cannula 112.

### F. Determining Access Point and Trajectory

As described above, the treatment procedure may be monitored by an intraoperative visualization by medical imaging (e.g., fluoroscopic imaging, ultrasonic imaging, CT imaging, or MRI imaging). Procedural risk can occur in pedicle access as a physician utilizes the medical images to traverse the pedicle and to avoid critical structures in the spine. It usually takes time for a physician to perfect his/her skills for pedicle access. However, mechanical, electronic, and/or software tools can be used to help physicians navigate pedicle access safely and more efficiently, to reduce the learning curve, and to provide safer and more accurate instrument placement.

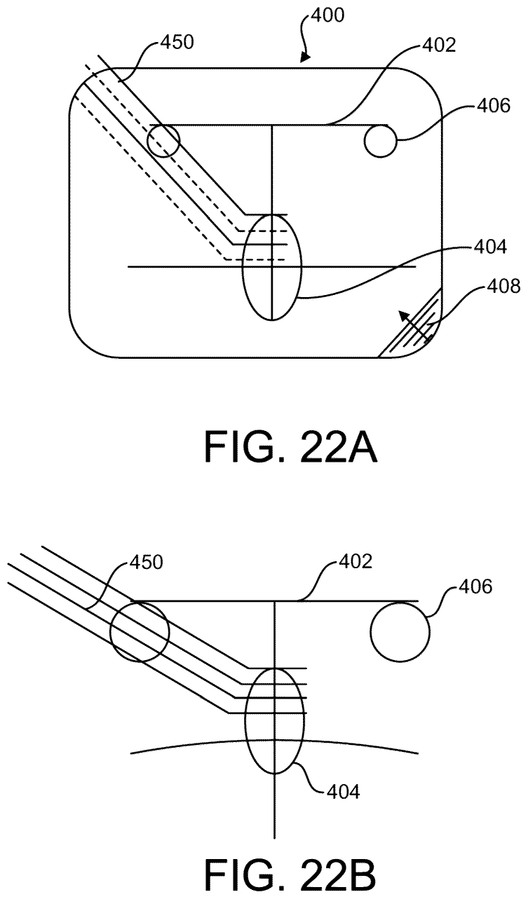

With reference to FIG. 22, in some embodiments, an adhesive-backed template overlay or sticker that has a printed pattern or markings (e.g., radiopaque pattern or markings) is placed on skin of the access area. In some embodiments, the template overlay or sticker is customized for a particular vertebra based on medical imaging of a portion of a particular patient's spine. The adhesive-backed template overlay or sticker can allow a physician to align respective markings of the template overlay or sticker with certain anatomical landmarks along the spine (e.g., vertebral endplate, spinous process, lateral borders of the pedicle, etc.) revealed in the medical (e.g., radiographic) imaging (e.g., a true anterior-posterior view). Such an aligned template overlay or sticker can more accurately pinpoint an incision point for pedicle access or other bone access. In some embodiments, the template of markings can be transferred to the skin of the access area by a transferable backing layer of the sticker (e.g., after introducer placement). In FIGS. 22A and 22B, radiopaque markings, including upper and/or lower vertebral endplate markings 402, spinous process markings 404, and/or pedicle markings 406, are printed on a backside of a sticker 400, which has a transferrable backing layer. The circles and/or ovals may also represent openings in the template or sticker. A top layer of the sticker 400 may be transparent so that the printed markings 402 are visible from the front side. When the sticker 400 is aligned with certain landmarks along the spine that are revealed by radiographic imaging, and is placed to adhere to the skin of the patient, the top layer of the sticker 400 can be peeled off by pulling a corner 408 of the sticker 400. After that, markings 402 are left on the skin as part of the transferrable backing layer to guide the physician's access into the vertebral body (e.g., transpedicular access). The trajectory lines 450 may be markings that represent various trajectory paths of the access tools (e.g., introducer cannula) through a pedicle to reach a target treatment region that a physician may choose from based on pre-operative measurements. The pre-operative measurements may be calculated manually or via an automated software program based on pre-operative medical imaging. For example, the pre-operative measurements may include vertebral body height. The target vertebral body treatment location may be predetermined based on a particular vertebral level or segment. For example, for lumbar vertebrae the target vertebral body treatment location may be 50% of the vertebral height.

In some embodiments, the template overlay or sticker may be applied to a back of the patient to align with anatomical landmarks based on the medical imaging and an optimal trajectory line of the trajectory lines 450 may be selected based on a determination of the line that dissects the pedicle projection properly (thus accounting for geometric bone variability). A skin access point would be along the selected line and would vary based on tissue thickness of the patient's back (e.g., back fat).

After identifying the optimal trajectory line, a physician or other clinician could switch the intraoperative medical imaging (e.g., C-arm fluoroscope) to an oblique view and identify the skin access point along the optimal trajectory line. The physician or other clinician could then insert the introducer cannula 112 or other access instrument along the trajectory line, ensuring that the angle is matched to the trajectory line to help reduce variability in access and to help reduce steering requirements during insertion of the curved cannula assembly 130, as described above. In accordance with several embodiments, use of the template overlay or sticker described herein may reduce two axes of rotation as variables.

In some embodiments, the printed markings on the adhesive backed sticker 400 may comprise radiopaque ink or metal deposition (e.g., via sputtering and/or photoetching) to allow for viewing radiographically during the procedure. In this way, the printed markings can be visible and interposed on the important landmarks of the spine in the radiographic or other medical imaging. In some embodiments, the printed markings can account for varying tissue thickness (e.g., body fat) to reduce time in determining proper entry position. In some embodiments, a kit that includes a variety of templates or stickers having printed markings can be provided to allow for selection during a procedure. In some embodiments, a set of template overlays or stickers may include appropriate markings for different sized vertebral bodies (e.g., vertebral bodies of different heights, such as 15 mm, 20 mm, 25 mm, etc.). The adhesive backed sticker can be a single-use sticker or can be reusable, for example, remaining on the patient for a subsequent procedure or procedures (e.g., treatment of different vertebral levels). In some embodiments, the device can be custom printed for each patient based on pre-operative imaging or planning software.

FIG. 22A may represent a template overlay or sticker for a larger vertebral body height (e.g., 25 mm) and FIG. 22B may represent a template overlay or sticker for a smaller vertebral body height (e.g., 20 mm). The trajectory paths in FIG. 22B are not as steep as the trajectory paths in FIG. 22A due to the reduced height. The trajectory paths may alternate between solid and dashed lines to help differentiate or may all be solid or have other patterns.

In some embodiments, a grid may be used that includes optional points of access and the physician or other clinical professional may be instructed to access at certain regions of the grid (e.g., based on pre-operative and/or intraoperative imaging).

Prior to or during a treatment procedure, software can be used to calculate the optimal pedicle access trajectory. In one embodiment, the software uses image recognition, machine learning or artificial intelligence algorithms or techniques to find an optimal path through a pedicle or other bone access path. In some embodiments, a physician or other medical professional can adjust the trajectory manually to find the optimal path. In some embodiments, the software establishes a coordinate system and outputs coordinates for certain spine landmarks (e.g., the L5 spinous process), for example, x coordinate, y coordinate, elevation angle and/or azimuth angle, that the physician or other medical professional can then use to guide introduction of the introducer tools for pedicle access. In some embodiments, the software can export the coordinates to another system, for example, to shine an LED light or laser overlay to show the point of entry directly on the patient.

In accordance with several embodiments, pedicle access may be guided by use of a pair of collimated radiopaque rings. The collimated rings may be formed by radiopaque markings or features that are spaced apart. The collimated rings may be integrated into an access instrument, such as introducer cannula 112 or may be included in a separate angulation element through which the access instrument may be inserted or that can be removably or fixedly attached to the access instrument (e.g., introducer cannula 112). In some embodiments, the rings include a proximal c-ring and a distal smaller ring to aid in introducer assembly trajectory alignment when attempting to match an angle of a C-arm fluoroscope or other imaging device. In some embodiments, the two radiopaque markers (e.g., rings) could be a la carte and snap onto an existing introducer assembly. In some embodiments, the radiopaque markers (e.g., rings) are molded into the handle of the introducer cannula or one into the handle and one along a distal end portion of the elongate tube of the introducer cannula. A difference in diameter between the two rings can aid an operator in developing a sense of depth perception when viewing a 2D fluoroscopic image and help the operator understand which direction to move the handle of the introducer cannula to bring the trajectory into a desired alignment (e.g., for transpedicular access). In some embodiments, a distal ring could simply slide over the introducer cannula prior to access and rest on the patient's back, thus not requiring a larger incision and also providing the greatest distance between the two rings for increased sensitivity. In some embodiments the distal ring could also serve to provide overdrive protection by locking onto the introducer cannula and resisting advancement by its physical size relative to the incision in the skin. In some embodiments, the distal ring could have adhesive to help secure to the skin, and use a series of different mechanisms (e.g., adhesive, collet, c-clamp, set screw, etc.) to clamp or otherwise couple to the introducer cannula for overdrive protection. In some embodiments the proximal marker is a ring, and in other embodiments it has a non-uniform shape to provide rotational alignment when using a bevel introducer. Because the collimated rings can be viewed on intraoperative medical imaging (e.g., fluoroscopic imaging), the collimated rings can be used to guide access through bone (e.g., through a pedicle). For example, the intraoperative medical imaging equipment (e.g., C-arm fluoroscope) can be rotated to an oblique view that images along a predetermined pedicle access angle. The operator can then move the introducer cannula until the collimated rings are aligned (e.g., a smaller ring is concentrically aligned with a larger ring) and then insert the introducer while maintaining the alignment. The introduction could be performed manually or robotically.

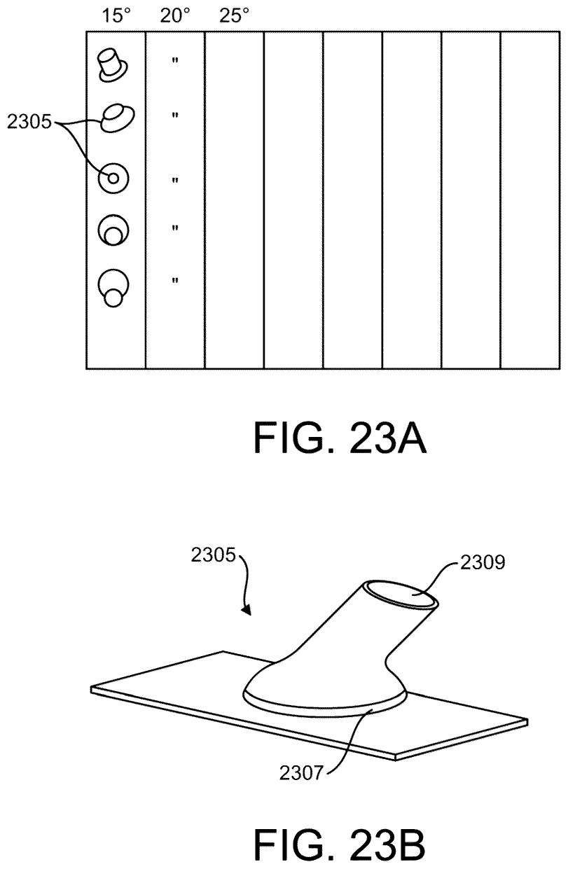

With reference to FIG. 23A, in some embodiments, a template 2300 to facilitate guided pedicle access may include raised 3D features or elements (e.g., plastic “mole hills” or protuberances) that may be injection molded or 3D printed, with different raised 3D features or elements 2305 provided for varying azimuth angles. FIG. 23B shows a close-up side view of one of the raised 3D features or elements 2305. The raised 3D features or elements 2305 may include a larger radiopaque ring 2307 at a base of the raised 3D feature or element and a smaller radiopaque ring 2309 at a raised position spaced apart from the base. Once a particular azimuth angle is determined, the adhesive portion (e.g., detachable strip) with the set of raised 3D features or elements 2305 for the determined azimuth angle can be removed from the template 2300 and placed on the patient. The physician or other clinician may select which one of the set of raised 3D features or elements 2305 is best based on an oblique view set by the predetermined pedicle access angle (e.g., azimuth angle). When placed on a patient, the template 2300 can set or define a proper access angle or trajectory for the access tools (e.g., introducer assembly), that may be pre-planned by the physician, thereby taking away one angle of rotation from the access equation. For example, based on pre-operative planning, a physician or operator can slide an introducer or other access tool through the angled raised 3D feature or device 2305 of the template 2300. In some embodiments, the template 2300 can include a self-adhesive to stick or adhere to the skin of the patient so as to mark one or more entry points and to aid in tool support. In some embodiments, the template 2300 (or one of the raised 3D features or devices 2305) can be attached to the access instrument (e.g., introducer cannula 112) and provide a pedicle stop to prevent unintended instrument advancement later in the procedure.

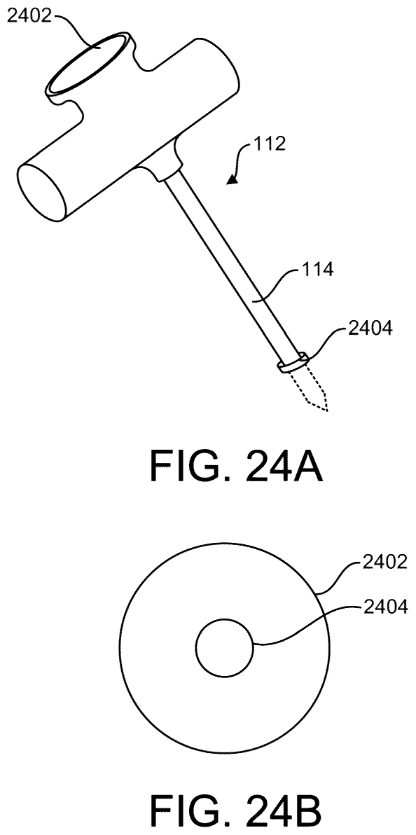

FIG. 24A illustrates an embodiment in which an introducer cannula itself includes the collimated radiopaque rings. The introducer cannula 112 includes a proximal ring 2402 located along an upper handle portion and a distal ring 2404 on a distal end portion of the introducer tube 114. The proximal ring 2402 and distal ring 2404 may be positioned at other locations as well. As shown, the proximal ring 2402 is larger than the distal ring 2404. FIG. 24B illustrates an example of how the distal ring 2404 and the proximal ring 2402 may be aligned on an oblique fluoroscopic view, as discussed above.

In some embodiments, an electromechanical device for pedicle access is attached to an access instrument (e.g., introducer cannula or introducer assembly) to identify or relay elevation and azimuth angle (and possibly other measurements or indicators) to the physician or other medical professional. In some embodiments, output from the software described above may be transmitted or entered into the electromechanical device to provide guidance as to when the operator has the device at an optimal entry angle (e.g., the angle determined in pre-planning by the software). In some embodiments, the device is fixedly attached (e.g., screwed to) or removably attached (e.g., clamped onto) to an instrument. In some embodiments, the device may be magnetically coupled to the instrument, either directly to a ferromagnetic surface or to itself in a clamshell-like design. In some embodiments, the device is first registered to the patient by aligning the instrument to known anatomic locations in the field of view to establish a virtual coordinate system before getting guidance on how to locate the instrument for pedicle access.

### G. Conclusion

In some implementations, the system comprises various features that are present as single features (as opposed to multiple features). For example, in one embodiment, the system includes a single radiofrequency generator, a single introducer cannula with a single stylet, a single curved cannula with a single J-stylet, a single radiofrequency energy delivery device or probe, and a single bipolar pair of electrodes. A single thermocouple (or other means for measuring temperature) may also be included. Multiple features or components are provided in alternate embodiments.

In some implementations, the system comprises one or more of the following: means for tissue modulation (e.g., an ablation or other type of modulation catheter or delivery device), means for monitoring temperature (e.g., thermocouple, thermistor, infrared sensor), means for imaging (e.g., MRI, CT, fluoroscopy), means for accessing (e.g., introducer assembly, curved cannulas, drills, curettes), means for controlling advancement within bone (e.g., pedicle stop, adjustable hypotube, surface features), means for guiding access (e.g., access templates, stickers or overlay templates with markings, etc.).

Although certain embodiments and examples have been described herein, aspects of the methods and devices shown and described in the present disclosure may be differently combined and/or modified to form still further embodiments. Additionally, the methods described herein may be practiced using any device suitable for performing the recited steps. Further, the disclosure (including the figures) herein of any particular feature, aspect, method, property, characteristic, quality, attribute, element, or the like in connection with various embodiments can be used in all other embodiments set forth herein. The section headings used herein are merely provided to enhance readability and are not intended to limit the scope of the embodiments disclosed in a particular section to the features or elements disclosed in that section.

While the embodiments are susceptible to various modifications, and alternative forms, specific examples thereof have been shown in the drawings and are herein described in detail. It should be understood, however, that the embodiments are not to be limited to the particular forms or methods disclosed, but to the contrary, the embodiments are to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “applying thermal energy” include “instructing the applying of thermal energy.”

Various embodiments of the disclosure have been presented in a range format. It should be understood that the description in range format is merely for convenience and brevity and should not be construed as an inflexible limitation on the scope of the invention. The ranges disclosed herein encompass any and all overlap, sub-ranges, and combinations thereof, as well as individual numerical values within that range. For example, description of a range such as from 5 to 15 minutes should be considered to have specifically disclosed subranges such as from 5 to 10 minutes, from 10 to 15 minutes, etc., as well as individual numbers within that range, for example, 5, 6, 7, 8, 9, 10, 11, 12, 13, 14, 15, 7.5 and any whole and partial increments therebetween. Language such as “up to,” “at least,” “greater than,” “less than,” “between,” and the like includes the number recited. Numbers preceded by a term such as “about” or “approximately” include the recited numbers. For example, “about 10” includes “10.” For example, the terms “approximately”, “about”, and “substantially” as used herein represent an amount close to the stated amount that still performs a desired function or achieves a desired result.

What is claimed is:

1. A medical device for forming a channel into a bone, the bone having an outer surface, comprising:
an introducer tube having a distal end and a proximal end disposed in a longitudinal direction;
a handle, the handing coupled with the proximal end of the introducer tube, wherein an internal channel is formed through the introducer tube and the handle, the internal channel configured to receive another longitudinal device from the handle to the distal end of the introducer tube; and
an edge formed at the distal end of the introducer tube; a distance from the edge to a tip of the distal end of the introducer tube forming a distal end length, the distal end length being greater than 0;
wherein the edge is configured to engage with the outer surface of the bone.

2. The device of claim 1, wherein the edge is a ringed edge formed around the lateral surface of the introducer tube.

3. The device of claim 1, wherein the edge is formed on an enlarged portion of the introducer tube.

4. The device of claim 1, wherein the edge is formed on a block coupled to the introducer tube.

5. The device of claim 1, wherein the edge is formed by a distal end of a hypotube sleeved on the introducer tube.

6. The device of claim 5, wherein a proximal end of the hypotube is coupled to the handle.

7. The device of claim 6, wherein the distal end length is adjustable by moving the hypotube longitudinally.

8. The device of claim 6, wherein the edge has a blunt curvature.

9. The device of claim 6, wherein the edge is partially bullet shaped.

10. The device of claim 6, wherein the edge is beveled.

11. The device of claim 7, wherein the longitudinal movement of the hypotube is achieved by rotating a wheel feature coupled to the handle, the wheel feature rotating around the introducer tube.

12. The device of claim 11, wherein the handle comprises an internal channel formed in the longitudinal direction, the internal channel having an internal thread, the proximal end of the hypotube having an external thread formed thereon configured to mate with the internal thread, wherein the proximal end of the hypotube is coupled to the wheel feature in a manner so that turning the wheel feature causes the hypotube to rotate but allows the hypotube to move freely longitudinally.

13. The device of claim 12, wherein the external thread is formed on the hypotube by insert molding.

14. The device of claim 11, wherein the handle comprises a protruded portion disposed in the longitudinal direction with an external thread formed thereon, wherein the hypotube is coupled with a wheel feature, the wheel feature having a hollowed extension disposed in the longitudinal direction with an internal thread formed therein, wherein the external thread on the protruded portion of the handle is configured to mate with the internal thread formed in the hollow extension of the wheel feature.

15. The device of claim 14, wherein the coupling between the hypotube and the wheel feature is a permanent attachment, wherein rotating the wheel feature causes the hypotube to move in the longitudinal direction.

16. The device of claim 15, wherein the attachment between the hypotube and the wheel feature is accomplished by insert molding.

17. The device of claim 15, wherein the attachment between the hypotube and the wheel feature is accomplished by interference fit.

18. The device of claim 15, wherein the attachment between the hypotube and the wheel feature comprises an adhesive.

19. The device of claim 14, wherein the coupling between the hypotube and the wheel feature allows the hypotube to move in the longitudinal direction, wherein a resilient ring is disposed inside the hollowed space of the hollowed extension of the feature and outside of the hypotube, and wherein rotating the wheel feature in one direction causes the resilient ring to be squeezed against the outer surface of the hypotube to stop the movement of the hypotube in longitudinal direction.

20. A system for performing radiofrequency (RF) ablation of one or more nerves within a vertebral body, comprising:
a device of any of claims 1;
a straight stylet, the straight style is configured to be received into the internal channel formed in the device;
a curved cannula assembly comprising a curved cannula and a J-stylet, wherein the curved cannula comprises a second internal channel formed therethrough configured to receive another longitudinal device, and wherein the curved cannula assembly is configured to be received into the internal channel formed in the device; and
a radiofrequency (RF) probe, the RF probe configured to be received into the second internal channel of curved cannula;
identifying a skin entry point positioned along a radiopaque trajectory line of the template overlay adhesive that properly dissects a pedicle projection;
inserting and advancing an introducer cannula through the skin entry point and through a pedicle while matching a trajectory angle of the introducer cannula with the radiopaque trajectory line under fluoroscopic guidance.
